SEQUENCE LISTING (3 sequences)

SEQ ID NO: 1 - protein, 142 aa, Homo sapiens
MGLKLNGRYISLILAVQIAYLVQAVRAAGKCDAVFKGFSDCLLKLGDSMANYPQGLDDKTNIKTVCTYWEDFHSCTVTALTDCQEGAKDMWDKLRKESKNLNIQGSLFELCGSGNGAAGSLLPAFPVLLVSLSAALATWLSF

SEQ ID NO: 2 - DNA, 426 bp, Artificial Sequence
  Description of Artificial Sequence Synthetic polynucleotide
atgggcctgaaactgaacggccgctatattagcctgattctggcggtgcagattgcgtatctggtgcaggcggtgcgcgcggcgggcaaatgcgatgcggtgtttaaaggctttagcgattgcctgctgaaactgggcgatagcatggcgaactatccgcagggcctggatgataaaaccaacattaaaaccgtgtgcacctattgggaagattttcatagctgcaccgtgaccgcgctgaccgattgccaggaaggcgcgaaagatatgtgggataaactgcgcaaagaaagcaaaaacctgaacattcagggcagcctgtttgaactgtgcggcagcggcaacggcgcggcgggcagcctgctgccggcgtttccggtgctgctggtgagcctgagcgcggcgctggcgacctggctgagcttt

SEQ ID NO: 3 - RNA, 426 nt, Artificial Sequence
  Description of Artificial Sequence Synthetic polynucleotide
augggccugaaacugaacggccgcuauauuagccugauucuggcggugcagauugcguaucuggugcaggcggugcgcgcggcgggcaaaugcgaugcgguguuuaaaggcuuuagcgauugccugcugaaacugggcgauagcauggcgaacuauccgcagggccuggaugauaaaaccaacauuaaaaccgugugcaccuauugggaagauuuucauagcugcaccgugaccgcgcugaccgauugccaggaaggcgcgaaagauaugugggauaaacugcgcaaagaaagcaaaaaccugaacauucagggcagccuguuugaacugugcggcagcggcaacggcgcggcgggcagccugcugccggcguuuccggugcugcuggugagccugagcgcggcgcuggcgaccuggcugagcuuu

(19) United States
(12) Patent Application Publication
(10) Pub. No.: US 2024/0366660 A1
(43) Pub. Date: Nov. 7, 2024

(54) Delivery of Cellular Material and Other Material as a Dry Powder

(71) Applicant: Pinata Holding Inc., Northvale, NJ (US)

(72) Inventors: Michael OGBURN, Lake Forest, CA (US); Christopher PRICE, Monroe Township, NJ (US)

(21) Appl. No.: 18/558,215

(22) PCT Filed: May 4, 2022
(86) PCT No.: PCT/US2022/027607

§ 371 Date: Oct. 31, 2023

Related U.S. Application Data
(60) Provisional application No. 63/184,410, filed on May 5, 2021.

(51) Int. Cl. A61K 35/12; A61K 9/50; A61K 45/06

(52) U.S. Cl. CPC A61K 35/12; A61K 9/5036; A61K 45/06

(57) ABSTRACT

Provided herein are pharmaceutical compositions, kits comprising pharmaceutical compositions, methods of treating and preventing disease, and methods of making compositions and kits described herein. The pharmaceutical compositions described herein are powdery pharmaceutical compositions. The powdery pharmaceutical compositions described herein can contain cellular material and can be microencapsulated to increase preservation of the cellular material. The powdery pharmaceutical compositions can be administered by numerous methods, such as by inhalation administration or oral administration.

### CROSS REFERENCE TO RELATED APPLICATIONS

This application claims the benefit of U.S. Provisional Application No. 63/184,410, filed May 5, 2021 the disclosure of which is incorporated herein by reference in its entirety.

### INCORPORATION BY REFERENCE

All publications, patents, and patent applications herein are incorporated by reference to the same extent as if each individual publication, patent, or patent application was specifically and individually indicated to be incorporated by reference. In the event of a conflict between a term herein and a term in an incorporated reference, the term herein controls.

### SUMMARY

Certain aspects of the disclosure herein pertain to spray drying cellular material that is at least partially encapsulated. In such aspects, the product may be a powdery composition comprising a plurality of spray dried particles, each particle of the plurality of spray dried particles comprising a eukaryotic cell, substantially encapsulated in a coating material, wherein: a) each particle of the plurality of spray dried particles individually has a particle diameter ranging from about 5 micrometers to about 100 micrometers, as measured by a particle analyzer using laser diffraction; b) the eukaryotic cell is a human cell; c) about 1% to 100% of the eukaryotic cells in the plurality of particles are alive; and d) the coating material comprises a hydroxypropyl methylcellulose (HPMC), a hydroxypropyl methylcellulose acetate succinate (HPMCAS), a cyclodextrin, a maltodextrin, a povidone, a copovidone, a trehalose, or any combination thereof. In some instances, the powdery substances may further comprise a pharmaceutically acceptable: excipient, diluent, or carrier. In some instances, wherein the pharmaceutically carrier is an excipient, the excipient may be in particle form, wherein at least a portion of the particles of the pharmaceutically acceptable excipient individually have a particle diameter ranging from about 50 micrometers to about 200 micrometers, as measured by a particle analyzer using laser diffraction. In some cases, the plurality of spray dried particles as described above may be within a pharmaceutically acceptable excipient which is admixed into a substantially homologous mixture. In some instances, the pharmaceutically acceptable excipient may comprise a carbohydrate, an alginate, povidone, a carbomer, a flavor, a natural gum, a silicone, an alcohol, a butter, a wax, a fatty acid, a preservative, a pharmaceutically acceptable salt of any of these, or any combination thereof.

In some instances, the powdery composition as discussed above may comprise from about 1 mg to about 200 mg of eukaryotic cells. In some cases, the powdery composition as discussed above may comprise from about 103 eukaryotic cells to about 1012 eukaryotic cells.

In the aforementioned instances, the eukaryotic cell may comprise an adult stem cell, an embryonic stem cell, a blood stem cell, a neural stem cell, an epithelial stem cell, a skin stim cell, mesenchymal stem cell, a very small embryonic-like stem cell (VSELs), a peripheral blood stem cell, (blastomeres), an induced pluripotent stem cell, an epithelial cell, an endothelial cells a fat cell, a white blood cell, a nerve cell, a platelet, a skeletal cell, a cartilage cell or a red blood cell. In some instances as applied above, the eukaryotic cell may comprise at least about 1% by weight to about 99% of the overall powdery pharmaceutical composition.

Also disclosed herein are powdery compositions comprising a plurality of spray dried particles, each particle of the plurality of spray dried particles comprising a cellular component or derivative thereof, substantially encapsulated in a coating material, and wherein: a) each particle of the plurality of spray dried particles individually has a particle diameter ranging from about 1 micrometer to about 100 micrometers or about 1 micrometer to about 10 micrometers, as measured by a particle analyzer using laser diffraction, and b) the coating material comprises a hydroxypropyl methylcellulose (HPMC), a hydroxypropyl methylcellulose acetate succinate (HPMCAS), a cyclodextrin, a maltodextrin, a povidone, a copovidone, a trehalose, or any combination thereof. In some instances, the cellular component or derivative thereof may comprise a protein having at least 75% homology and at least 75% length to SEQ ID NO: 1. In some instances, the homology may comprise at least 90% sequence homology to SEQ ID NO: 1. In some instances, concerning either powdery composition as described above, the composition may be contained within a capsule or a container.

In some instances, the plurality of spray dried particles described above and herein are encapsulated by one or more additional coating materials. In some cases, the coating materials are the same coating material. In some cases, the coating materials are different coating materials. In some instances, the powdery composition as described above may be a pharmaceutical composition optionally in unit dose form. In some instances, the powdery composition may be for ocular use, intravenous use, subcutaneous use, topical use, oral use, or a combination thereof.

In the case of oral use, the plurality of spray dried particles may be at least partially surrounded by a first capsule, a second capsule, or both; and wherein the first capsule is surrounded by the second capsule. In such an instance, the powdery substance may further comprise a second active ingredient, or a pharmaceutically acceptable salt thereof in unit dose form. In some instances concerning the second active ingredient, this ingredient may be at least partially surrounded by the first capsule, the second capsule or both. Still further, the second active ingredient may comprise particles, wherein the particles are at least partially encapsulated by a coating material and wherein the particles at least partially encapsulated by the coating material are spray dried.

Regarding the first capsule and the second capsule, in some cases one or both may comprise a capsule coating. Still further, such a capsule coating may at least partially control active ingredient release. In instances wherein the capsule coating at least partially controls active ingredient release, the capsule coating may comprise an enteric coating, a time release coating, a pH dependent coating, a delayed release coating, an extended-release coating, or a combination thereof. In some instances regarding the first and the second capsule, they may be formulated to deliver their contents at different locations in the gastrointestinal system. In some instances, the first capsule and the second capsule are formulated to deliver their contents at about the same location in the gastrointestinal system. In further instances regarding the first and/or second capsule, the capsule may comprise a capsule band added to the first capsule, the second capsule, or both and wherein the capsule band at least partially seals the capsule. In further instances regarding the first and/or second capsule, either or both may comprise a hydroxypropyl methylcellulose (HPMC). In some instances, the first capsule is size: 000, 00, 0, 1, 2, 3, 4, or 5. In some instances, the second capsule is size: 000, 00, 0, or 1.

In some instances regarding a eukaryotic cell as described above, the eukaryotic cell or the cellular component substantially encapsulated in the coating material may have a particle diameter ranging from about 10 micrometers to about 100 micrometers or about 20 micrometers to about 80 micrometers.

In some instances the powdery formulation may be for inhaled or intranasal use. The powdery formulation may be contained within an inhaler unit. The inhaler unit may comprise the powdery composition in the capsule. In such instances, the capsule may be about one quarter to about one half, by volume, filled with the powdery composition. In such cases, a portion of the capsule not containing the powdery composition may comprise a gas that at least partially comprises an inert gas. In instances wherein the powdery composition is within an inhaler unit, the capsule is size may be: 000, 00, 0, 1, 2, 3, or 4. In particular instances, the capsule is size 3. In some instances, the plurality of spray dried particles comprising the eukaryotic cell substantially encapsulated in the coating material individually may have a particle diameter ranging from about 5 micrometers to about 30 micrometers, 10 micrometers to about 20 micrometers, or about 5 micrometer to about 15 micrometers. Still further, the plurality of spray dried particles comprising the cellular component substantially encapsulated in the coating material individually have a particle diameter ranging from about 1 micrometers to about 30 micrometers, 10 micrometers to about 20 micrometers, or about 1 micrometer to about 10 micrometers.

In some aspects of the disclosure, the powdery compositions as described above may be included in a kit at least in part in a packaging.

Other aspects of the disclosure pertain to a method of treating or preventing a disease or condition in a subject in need thereof, comprising treating or preventing the disease or condition by administering a therapeutically effective amount of a powdery composition as described above to the subject in need thereof. In some instances, the administration may be conducted one, twice, three, or four times per day.

Regarding a disease or condition, in some instances, the disease or condition is selected from the group consisting of: a blood related disease, a degenerative disease, a heart failure, a spinal cord injury, Type 1 diabetes, Type 2 diabetes, Parkinson's disease, Alzheimer's disease, amyotrophic lateral sclerosis, a stroke, a burn, a skin damage, an osteoarthritis, an arthritis, a diabetic cognitive dysfunction in type 2 diabetes, astrogliosis associated with diabetes, a depression, a schizophrenia, a genetic disease, a chronic obstructive pulmonary disease (COPD), a chronic bronchitis, an emphysema, a reactive airway disease, an infectious disease, a muscular injury, a skeletal injury, an inflammatory disease, an allergy, and a neuronal injury.

In such instances regarding administration, the powdery composition may be administered as needed, or for about: one day, two days, three days, four days, five days, six days, a week, two weeks, three weeks, a month, two months, three months, four months, five months, six months, seven months, eight months, nine months, ten months, eleven months, a year, or chronically. In some instances, the amount of the eukaryotic cell or the cellular material ranges from about 1 mg to about 200 mg. In some instances, the amount of the eukaryotic cell or the cellular material ranges from about 1 mg to about 10 mg.

Still further, in some instances pertaining to a method of treating or preventing a disease or condition, the treatment may comprise administration of a second therapeutic or pharmaceutically acceptable salt thereof. In instances regarding a second therapeutic or pharmaceutically acceptable salt thereof, the administration may be concurrently or consecutively. Such a second therapeutic or pharmaceutically acceptable salt thereof may be comprised in the powdery formulation. In other instances, the second therapeutic or the pharmaceutically acceptable salt thereof is not comprised in the powdery formulation.

The methods of treatment or prevention of a disease or condition may include diagnosing the subject with the disease or condition. In some instances, diagnosing comprises employing an in vitro diagnostic. The in vitro diagnostic may be a companion diagnostic.

Also disclosed herein are methods of making a powdery composition as describe above, comprising contacting the eukaryotic cell and the coating material in a solution, wherein the coating material comprises the hydroxypropyl methylcellulose (HPMC), the hydroxypropyl methylcellulose acetate succinate (HPMCAS), the cyclodextrin, the maltodextrin, the povidone, the copovidone, a trehalose, or any combination thereof and spray drying the eukaryotic cell, the coating material and the solvent to form a substantially encapsulated eukaryotic cell.

Regarding contacting the eukaryotic cell, the contacting may comprise contacting the cellular component and the coating material in a solution, wherein the coating material comprises the hydroxypropyl methylcellulose (HPMC), the hydroxypropyl methylcellulose acetate succinate (HPMCAS), the cyclodextrin, the maltodextrin, the povidone, the copovidone, a trehalose, or any combination thereof and spray drying the cellular component, the coating material and the solvent to form a substantially encapsulated cellular component.

In other aspects of the disclosure, a powdery composition may comprise a plurality of spray dried particles, each particle of the plurality of spray dried particles comprising a cellular component or derivative thereof, substantially encapsulated in a coating material, wherein: a) each particle of the plurality of spray dried particles individually has a particle diameter ranging from about 1 micrometer to about 100 micrometers or about 1 micrometer to about 10 micrometers, as measured by a particle analyzer using laser diffraction; b the cellular component or derivative thereof comprises a protein having at least 75% homology and at least 75% length to SEQ ID NO: 1; and c) the coating material comprises a hydroxypropyl methylcellulose (HPMC), a hydroxypropyl methylcellulose acetate succinate (HPMCAS), a cyclodextrin, a maltodextrin, a povidone, a copovidone, a trehalose, or any combination thereof.

### BRIEF DESCRIPTION OF THE DRAWINGS

FIG. 1A shows a dry powder inhaler device for delivery of a powdery pharmaceutical composition to the lung alveolar and FIG. 1B shows a nasal inhaled device for intranasal delivery of a powdery pharmaceutical composition to the lung alveolar.

FIG. 2 shows the method of use for the dry powder inhaler device for delivery of a powdery pharmaceutical composition.

FIG. 3 shows a spray drying manufacturing system comprising a closed spray drying chamber which receives a solution comprising an active ingredient microencapsulated in a polymer in a suitable solvent. The system generates dried microencapsulated particles from the solution comprising the microencapsulated active ingredient.

FIG. 4 shows a protective cap for a dry powder inhaler device.

FIG. 5 shows a rotatable mouthpiece of a dry powder inhaler device.

FIG. 6 shows a lower base chamber receptacle of a dry powder inhaler device.

FIG. 7 shows a lateral button operably connected to a sharp surface for use in a dry powder inhaler device for piecing a capsule containing a dry powdery pharmaceutical composition.

FIG. 8 shows a base plate of a dry powder inhaler device.

FIG. 9 shows a dry powder inhaler device for delivery of a powdery pharmaceutical composition to the lung alveolar.

FIG. 10 shows the process of a spray dry system that creates a dry powder comprising the at least partially encapsulated cellular material (e.g., the at least partially encapsulated cells, the at least partially encapsulated one or more cellular components, or both).

### DETAILED DESCRIPTION

### Overview

Spray drying cellular material can be an efficient way to formulate a composition and administer a composition comprising at least partially encapsulated cells, at least partially encapsulated cellular components, or both. In some cases, a composition herein can be administered through oral ingestion for example, by a capsule-in capsule delivery system. A capsule-in-capsule system can be used to release a composition described herein to different (or the same) locations of the gastrointestinal tract. In some cases, an encapsulated cellular material can be introduced into the lungs via inhalation administration. In some cases, the time needed for the pharmaceutical to reach the blood stream can be significantly reduced via inhalation administration as compared to oral administration. Also described herein are methods of administering compositions described herein via an injection or intravenous administration, ophthalmic administration (e.g., eye drops), and topical administration.

Provided herein are methods of at least partially encapsulating cells, which can include live cells. Also provided herein are methods of at least partially encapsulating one or more cellular components. In some instances, a cellular material can comprise an at least partially encapsulated cell, an at least partially encapsulated cellular component, or both. In some cases, the cellular material can be in unit dose form. In some instances, cellular material can be in the form of a powdery composition.

In some instances, also provided herein are methods of administering the at least partially encapsulated cells, at least partially encapsulated one or more cellular components, or both to a subject. In some cases, the subject can have a disease or a condition.

In some instances, a cellular material described herein can comprise a pharmaceutical composition. For example, the powdery compositions described herein can be powdery pharmaceutical compositions. Provided herein are pharmaceutical compositions, kits comprising pharmaceutical compositions, methods of treating and preventing disease, and methods of making compositions and kits described herein. Pharmaceutical drugs described herein can be produced employing various methods to synthesize, manipulate, and administer particles. In some instances, the pharmaceutical compositions described herein are powdery compositions and can be produced by a spray drying process.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various aspects may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining”, “measuring”, “evaluating”, “assessing,” “assaying,” and “analyzing” are often used interchangeably herein to refer to forms of measurement, and include determining if an element may be present or not (for example, detection). These terms can include quantitative, qualitative or quantitative, and qualitative determinations. Assessing can be alternatively relative or absolute. “Detecting the presence of” includes determining the amount of something present, as well as determining whether it may be present or absent.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. In some cases, the subject can be a subject in need thereof, for example a subject in need of a treatment. The subject can be a mammal. In some instances, the subject can be an animal. In some instances, the subject can be a human. In some instances, the subject can be a dog, a cat, a horse, a farm animal, a cow, a goat, a bird, a pet, or a pig. The subject may be diagnosed or suspected of being at high risk for a disease. In some cases, the subject may not be necessarily diagnosed or suspected of being at high risk for the disease.

The term “substantially” or “essentially” can refer to a qualitative condition that exhibits an entire or nearly total range or degree of a feature or characteristic of interest. In some cases, substantially encapsulated can refer to near complete encapsulation of a substance or compound. For example, substantially encapsulated can comprise a particle that is at least about: 70%, 75%, 80%, 85%, 90%, 95%, 99%, or 100% encapsulated. In some cases, substantially can refer to at least about: 70%, 75%, 80%, 85%, 90%, 95%, 99%, or 100% of the total range or degree of a feature or characteristic of interest.

The term “at least partially” can refer to a qualitative condition that exhibits a partial range or degree of a feature or characteristic of interest. In some cases, at least partially encapsulated can refer to a partial encapsulation of a substance or compound. For example, at least partially encapsulated can comprise a particle that is at least about: 5%, 10%, 20%, 30%, 40%, 50%, 60%, 70%, 75%, 80%, 85%, 90%, 95%, 99%, or 100% encapsulated.

The term “in vivo” can be used to describe an event that takes place in a subject's body.

The term “ex vivo” can be used to describe an event that takes place outside of a subject's body. An “ex vivo” assay may not be performed on a subject. Rather, it can be performed upon a sample separate from a subject. An example of an “ex vivo” assay performed on a sample can be an “in vitro” assay.

The term “in vitro” can be used to describe an event that takes place contained in a container for holding laboratory reagent such that it can be separated from the living biological source organism from which the material may be obtained. In vitro assays can encompass cell-based assays in which cells alive or dead are employed. In vitro assays can also encompass a cell-free assay in which no intact cells are employed.

As used herein, the term “about” a number can refer to that number plus or minus 5% or 10% of that number. The term “about” a range can refer to that range minus 10% of its lowest value and plus 10% of its greatest value. The term “about” a range can refer to that range minus 5% of its lowest value and plus 5% of its greatest value. In some cases, the term “about” a number can refer to that number plus or minus 20% of that number. The term “about” a range can refer to that range minus 20% of its lowest value and plus 20% of its greatest value.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement may be observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

As used herein, the term “unit dose” or “dosage form” can be used interchangeably and can be meant to refer to pharmaceutical drug products in the form in which they are marketed for use, with a specific mixture of active ingredients and inactive components or excipients, in a particular configuration, and apportioned into a particular dose to be delivered. The term “unit dose” can also sometimes encompass non-reusable packaging, although the FDA distinguishes between unit dose “packaging” or “dispensing”. More than one unit dose can refer to distinct pharmaceutical drug products packaged together, or to a single pharmaceutical drug product containing multiple drugs and/or doses. The term “unit dose” can also sometimes refer to the particles comprising a pharmaceutical composition, and to any mixtures involved. Types of unit doses may vary with the route of administration for drug delivery, and the substance(s) being delivered. A solid unit dose can be the solid form of a dose of a chemical compound used as a pharmaceutically acceptable drug or medication intended for administration or consumption.

As used herein, the term “fine particle fraction” or “fine particle fraction from the emitted dose” can refer to the mass of active agent having an aerodynamic diameter below about: 5 μm, 6 μm, 7 μm, 8 μm, 9 μm, or 10 μm. In some instances, the cutoff size can be less than or equal to an aerodynamic diameter of about 5 μm. In some instances, the cutoff size can be less than or equal to an aerodynamic diameter of about 6.4 μm. In some instances, the cutoff size can be less than or equal to an aerodynamic diameter of about 7 μm or about 8 μm. In some instances, the fine particle fraction can be often used to evaluate the efficiency of aerosol deaggregation. In some cases, fine particle fraction can be the mass of active agent having an aerodynamic diameter below about: 5 μm, 6 μm, 7 μm, 8 μm, 9 μm, or 10 μm as a percentage of an emitted dose mass. For example, a composition described herein can have a fine particle fraction of at least about: 20%, 30%, 40%, 50%, 60%, 70%, 80%, or 90% upon aerosolization.

As used herein, a “dose” can refer to a measured quantity of a therapeutic agent to be taken at one time.

As used herein, “pharmaceutically acceptable salt” can refer to pharmaceutical drug molecules, which may be formed as a weak acid or base, chemically made into their salt forms, most frequently as the hydrochloride, sodium, or sulfate salts. Drug products synthesized as salts may enhance drug dissolution, boost absorption into the bloodstream, facilitate therapeutic effects, and increase its effectiveness. Pharmaceutically acceptable salts may also facilitate the development of controlled-release dosage forms, improve drug stability, extend shelf life, enhance targeted drug delivery, and improve drug effectiveness.

As used herein, “laser diffraction” can refer to a method for particle size analysis, which consists of scattering laser light off an assembly of particles, and collecting the scattered light using a spatial array of detectors. The signal from the detectors can be a pattern of scattered/diffracted light vs. angle. This pattern can result from many particles being illuminated by the laser light source at the same time, where all of their individual scattered/diffracted light rays mix together at each detector element.

As used herein, “particle size analyzer” can refer to an instrument for particle size analysis, particle size measurement, or simply particle sizing.

As used herein, “particle size analysis” can refer to the collective name of the technical procedures, or laboratory techniques which determines the size range, and/or the average (mean), median or mode size of the particles in a powder or liquid sample.

As used herein, “time to peak plasma concentration” can refer to the time required for a drug to reach peak concentration in plasma. Peak concentration in plasma can be usually defined as the plasma concentration that a drug achieves in a specified compartment or test area of the body after the drug has been administered and before the administration of a second dose.

As used herein, “HPLC” can refer to high-performance liquid chromatography (formerly referred to as high-pressure liquid chromatography), which is a technique in analytical chemistry used to separate, identify, and quantify each component in a mixture. HPLC can be a common technique used in pharmaceutical development, as it can be a method to ensure product purity.

As used herein, the terms “effective amount” or “therapeutically effective amount” of a drug used to treat a disease can be an amount that can reduce the severity of a disease, reduce the severity of one or more symptoms associated with the disease or its treatment, or delay the onset of more serious symptoms or a more serious disease that can occur with some frequency following the treated condition. An “effective amount” may be determined empirically and in a routine manner, in relation to the stated purpose.

As used herein, the term “substantially” can refer to a degree of deviation that is sufficiently small so as to not measurably detract from the identified property or circumstance. In some cases, the exact degree of deviation allowable may in some cases depend on the specific context.

The section headings used herein are for organizational purposes only and are not to be construed as limiting the subject matter described.

### Pharmaceutical Compositions

Disclosed herein are devices, systems and methods for producing, packaging, and delivering stable powdery pharmaceutical compositions to a subject in need thereof. In some cases, a pharmaceutical composition can comprise a cellular material. In some instances, a cellular material can comprise a supplement. In some cases, delivery can comprise delivery to the lungs via intranasal inhalation. In some cases, delivery can comprise a capsule-in-capsule formulation. In some embodiments, the pharmaceutical compositions can be spray dried. In those instances, the addition of an excipient carrier product to the active pharmaceutical powders prior to encapsulation can improve its stability and effective solubility. In some cases, an active ingredient as described herein can comprise a cell, a cellular component, or both.

Disclosed herein are powdery compositions. In some cases, a powdery composition can comprise a spray dried particle and a spray dried particle can comprise a eukaryotic cell or a cellular component substantially encapsulated in a coating material. In some cases, a powdery composition can comprise a plurality of spray dried particles, each particle of the plurality of spray dried particles comprising a eukaryotic cell or a cellular component, substantially encapsulated in a coating material. In some cases, each particle of the plurality of spray dried particles individually has a particle diameter ranging from about 1 micrometers to about 100 micrometers, or about 5 micrometers to about 100 micrometers as measured by a particle analyzer using laser diffraction. In some cases, the eukaryotic cell or the cellular component is a human cell or a cellular component of human origin. In some cases, about 1% to 100% of the eukaryotic cells in the plurality of particles are alive. In some instances, the coating material can comprise a hydroxypropyl methylcellulose (HPMC), a hydroxypropyl methylcellulose acetate succinate (HPMCAS), a cyclodextrin, a maltodextrin, a povidone, a copovidone or any combination thereof.

In some embodiments the compositions can comprise one or more of: an active ingredient or salts, excipients, and inactive ingredients. For example, a composition disclosed herein can comprise 1, 2, 3, 4, 5, 6, or more cellular materials. In some cases, a pharmaceutical composition can comprise particles. In some cases, particles can comprise an excipient (e.g. a pharmaceutically acceptable excipient) or an active ingredient. In some cases, the compositions can comprise a pharmaceutical composition. In some instances, a composition can comprise particles of a pharmaceutically acceptable excipient. In some instances, a composition can comprise particles comprising an active ingredient or a pharmaceutically acceptable salt thereof at least partially encapsulated in a coating material and wherein the particles at least partially encapsulated in the coating material are spray dried.

In some embodiments, a coating material can comprise a material added via a pharmaceutical coating process by which an essentially dry, outer layer of coating material can be applied to the surface of a dosage form. Coating dosage forms may be used to improve stability (light protection, moisture and gas barrier), facilitate administration, or modify the drug release behavior from the dosage form. The coating materials may be used to enable the immediate release of the drug, delay the release of the drug (such as in enteric coatings), or sustain the release of the drug from the dosage form over extended periods of time Coating materials may include film coating formulations, which usually contain a polymer, a plasticizer, a colorant, opacifier, a solvent, and a vehicle. In some cases, a coating material can refer to the coating material used in the coating of a particle of an active ingredient to create an encapsulated particle.

In some embodiments, a composition can comprise a mixture of particles described herein. In some instances, the particles can be mixed in a substantially homogenous mixture. In some instances, at least a portion of the particles of the pharmaceutically acceptable excipient can have a particle diameter ranging from about 50 μm (micrometers) to about 200 μm, as measured by a particle size analyzer using laser diffraction; at least a portion of the particles comprising an active ingredient or a pharmaceutically acceptable salt thereof at least partially encapsulated in a coating material can have a particle diameter ranging from about 1 μm (micrometer) to about 100 μm, 10 μm to about 50 μm, or from about 1 μm to about 200 μm, as measured by a particle size analyzer using laser diffraction.

In some embodiments, a composition herein can be in the form of capsule-in-capsule formulation. For example, substantially encapsulated spray dried particles comprising a cellular material can be at least partially surrounded by a first capsule, a second capsule, or both. In some cases, the first capsule can be surrounded by a second capsule. In some cases, the composition can further comprise a second active ingredient, or a pharmaceutically acceptable salt thereof in unit dose form, an excipient, or both. In some cases, a capsule can comprise a capsule coating. In some cases, a capsule coating can at least partially control active ingredient release.

In some embodiments, the particles comprising a first active ingredient (e.g., a first cellular material) can be comprised in a first capsule. In some instances, the particles comprising the second active ingredient (e.g., a second cellular material) can be comprised in a second capsule. In some instances, a first capsule can be comprised in a second capsule. In some instances, the particles comprising the second active ingredient can be released from about 1 min to about 20 min, about 10 min to about 60 min, about 20 min to about 120 min, about 1 hour to about 2 hours, about 1 hour to about 3 hours, about 1 hour to about 4 hours, about 1 hour to about 5 hours, about 1 hour to about 6 hours, about 1 hour to about 7 hours, about 1 hour to about 8 hours, about 1 hour to about 9 hours, about 1 hour to about 10 hours, about 5 hours to about 15 hours, about 5 hours to about 20 hours, about 5 hours to about 30 hours, about 5 hours to about 35 hours, about 10 hours to about 20 hours, about 10 hours to about 15 hours, about 10 hours to about 20 hours, about 10 hours to about 30 hours, about 10 hours to about 35 hours, about 20 hours to about 30 hours, or about 20 hours to about 35 hours earlier than the particles comprising the first active ingredient. In some instances, a composition comprised in the second capsule can be released from about 1 min to about 20 min, about 10 min to about 60 min, about 20 min to about 120 min, about 1 hour to about 2 hours, about 1 hour to about 3 hours, about 1 hour to about 4 hours, about 1 hour to about 5 hours, about 1 hour to about 6 hours, about 1 hour to about 7 hours, about 1 hour to about 8 hours, about 1 hour to about 9 hours, about 1 hour to about 10 hours, about 5 hours to about 15 hours, about 5 hours to about 20 hours, about 5 hours to about 30 hours, about 5 hours to about 35 hours, about 10 hours to about 20 hours, about 10 hours to about 15 hours, about 10 hours to about 20 hours, about 10 hours to about 30 hours, about 10 hours to about 35 hours, about 20 hours to about 30 hours, or about 20 hours to about 35 hours earlier than a composition comprised in the first capsule.

In some cases, in a human clinical trial, the powdery pharmaceutical composition, when inhaled into the lungs, can provide in at least part of the humans in the clinical trial a time to peak plasma concentration (Tmax) of the active ingredient or the salt thereof ranging from about 1 minute to about one hour, or from about 1 minute to about ten minutes. In some instances, the Tmax of the active ingredient or the salt thereof ranging from about 1 min to about 5 min, about 1 min to about 10 min, about 1 min to about 20 min, about 1 min to about 25 min, about 1 min to about 30 min, about 1 min to about 40 min, about 1 min to about 50 min, about 1 min to about 60 min, about 5 min to about 10 min, about 5 min to about 20 min, about 5 min to about 25 min, about 5 min to about 30 min, about 5 min to about 40 min, about 5 min to about 50 min, about 5 min to about 60 min, about 10 min to about 20 min, about 10 min to about 25 min, about 10 min to about 30 min, about 10 min to about 40 min, about 10 min to about 50 min, about 10 min to about 60 min, about 20 min to about 25 min, about 20 min to about 30 min, about 20 min to about 40 min, about 20 min to about 50 min, about 20 min to about 60 min, about 25 min to about 30 min, about 25 min to about 40 min, about 25 min to about 50 min, about 25 min to about 60 min, about 30 min to about 40 min, about 30 min to about 50 min, about 30 min to about 60 min, about 40 min to about 50 min, about 40 min to about 60 min, or about 50 min to about 60 min. In some instances, when inhaled into the lungs in a human clinical trial, the powdery pharmaceutical composition operates mechanistically such that in at least a portion of the humans in the clinical trial, a majority of the particles of the pharmaceutically acceptable excipient deposit onto the oropharynx.

In some embodiments, the weigh to weight ratio of: a) the particles of the pharmaceutically acceptable excipient and b) particles comprising an active ingredient or a pharmaceutically acceptable salt thereof at least partially encapsulated in a coating material ranges from about 1:1 to about 10000:1. In some instances, the weight to weight ratio of: a) the particles of the pharmaceutically acceptable excipient and b) particles comprising an active ingredient or a pharmaceutically acceptable salt thereof at least partially encapsulated in a coating material can range from about 1:1 to about 20:1, about 1:1 to about 15:1, about 1:1 to about 10:1, about 1:1 to about 5:1, about 1:1 to about 2:1, about 2:1 to about 20:1, about 2:1 to about 15:1, about 2:1 to about 10:1, about 2:1 to about 5:1, about 5:1 to about 20:1, about 5:1 to about 15:1, about 5:1 to about 10:1, about 10:1 to about 15:1, about 10:1 to about 20:1, about 15:1 to about 20:1, about 18:1 to about 25:1, or about 25:1 to about 30:1. In some instances, the weight to weight ratio of: a) the particles of the pharmaceutically acceptable excipient and b) particles comprising an active ingredient or a pharmaceutically acceptable salt thereof at least partially encapsulated in a coating material can be about: 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, or 30:1 In some instances, the weight to weight ratio of: a) the particles of the pharmaceutically acceptable excipient and b) particles comprising an active ingredient or a pharmaceutically acceptable salt thereof at least partially encapsulated in a coating material can range from about 1:1 to about 1:10, about 1:1 to about 1:8, about 1:1 to about 1:5, about 1:1 to about 1:2, about 1:2 to about 1:10, about 1:2 to about 1:8, about 1:2 to about 1:5, about 1:5 to about 1:10, about 1:5 to about 1:8, about 1:8 to about 1:10. In some instances, an active ingredient or a pharmaceutically acceptable salt thereof (e.g. a cellular material or the pharmaceutically acceptable salt thereof) can comprise at least about: 1%, 2%, 3%, 4%, 5%, 6%, 7%, 8%, 9%, or 10% by weight of a pharmaceutical composition.

In some instances, at least a portion of the particles of the first pharmaceutical excipient and particles comprising an active ingredient or a pharmaceutically acceptable salt thereof at least partially encapsulated in a coating material may not be covalently bound to each other.

### Active Ingredients and Pharmaceutically Acceptable Salts

An active pharmaceutical ingredient can be any substance or mixture of substances intended to be used in the manufacture of a drug (medicinal) product and that, when used in the production of a therapeutic, becomes an active ingredient of the therapeutic. Such substances can be intended to furnish pharmacological activity or other direct effect in the diagnosis, cure, mitigation, treatment, or prevention of disease or to affect the structure or function of the body. In some cases, an active ingredient can comprise a pharmaceutical compound, a cellular material, or both. In some cases, an active ingredient can comprise a pharmaceutical compound, a cellular material, or both. In some cases, a pharmaceutical compound can comprise an active ingredient.

In some embodiments, an active pharmaceutical ingredient or salt thereof can be formulated as oil emulsion. In some instances, an active pharmaceutical ingredient or salt thereof can be formulated as an oil, a liquid, or a gel. In some instances, an active pharmaceutical ingredient or salt thereof can be formulated as a substantially dry material. In some instances, the active pharmaceutical ingredient or salt thereof can be encapsulated in a coating material and can be spray dried.

In some embodiments, the disclosure provides for the creation and delivery of cellular material as a dry powder. The cellular material can be delivered as a dry powder drug utilizing different routes of administration such as inhalation, intranasal, oral, or reconstituted to be administered intravenously, by injection or using eye drops. In some instances, the advanced engineering and process automation can allow for enhanced methods of manufacturing to achieve consistent cellular material that can be used to treat patients.

In some instances, the active pharmaceutical ingredients may comprise biologic material (e.g., cellular material) or salts thereof. In certain instances, the cellular material may comprise one or more cells, one or more cellular components, or a mixture thereof. In some cases, a cellular component can comprise a cellular material. In some cases, a cellular component can comprise a derivative of a cellular component. In some cases, a cell, or a cellular component, or both may be isolated and/or purified. In some cases, wherein a cell or a cellular component is contemplated for use, the cell may comprise a eukaryotic cell such as a human cell, an animal cell, a plant cell, or a fungal cell. In some cases, a cell can comprise, a prokaryotic cell. In some cases, a cell can be a cultured cell, or a natural cell.

In some embodiments, a cell can be genetically altered. In some cases, a cell can comprise a chimeric antigen receptor (CAR) such as a CAR T-cell or a CAR NK cell. In some cases, a cell can comprise exogenous DNA or RNA. In some instances, a cell can be autologous or allogenic. In some instances, a cell can contain a transgene, a guide, for example a CRISPR guide, or an RNA editing guide, or any combination thereof. In some instances, a cell can contain a guide which can facilitate an epigenetic alteration. In some instances, a gene in one or more cells can be knocked out or suppressed. In some instances, a gene in one or more cells can be over expressed.

In certain instances, wherein the cell is a eukaryotic cell, the cell may be an immune cell (e.g., a lymphocyte), such as, but not limited to naïve T cells, Th17 T cells, Th1 T cells, Th2 T cells, THαβ T cells, Th9 T cells, T follicular helper T cells (Tfh), regulatory T cells (Treg), CD4+ T cells, cytotoxic T cells (CD8+), central memory T cell (Tcm), tissue resident memory T cells (Trm), virtual memory T cells, innate memory T cells, memory stem cells (Tscm), γδ T cells, natural killer (NK) cells, transitional B cells, naïve B cells, effector B cells, memory B cells, B1 cells, B2 cells, neutrophils, eosinophils, monocytes, macrophages, basophils, mast cells, dendritic cells, hybridoma cells, derivatives of these, and/or combinations of these.

In certain instances, wherein the cell is a eukaryotic cell, the cell may be a hematopoietic cell such as, but not limited to hematopoietic stem cells (CD34+), proerythroblasts, normoblast cells, promyelocyte cells, reticulocyte cells, erythrocyte cells, pre-erythrocyte cells, myeloblast cells, erythroblast cells, megakaryocyte cells, B cell progenitor cells, T cell progenitor cells, thymocyte cells, macrophage cells, mast cells, thrombocyte cells derivatives of these, and/or combinations of these.

In certain instances, wherein the cell is a eukaryotic cell, the cell may be a cell typically associated with the nervous system such as, but not limited to astrocytes (protoplasmic and fibrous), microglia, oligodendrocytes, and neurons ependymocytes, pinealocytes, inner pillar cells of organ of Corti, outer pillar cells of organ of Corti, inner phalangeal cells of organ of Corti, outer phalangeal cells of organ of Corti, border cells of organ of Corti, Hensen cells of organ of Corti, Schwann cells, satellite glial cells, enteric glial cells, derivatives of these, and/or combinations of these.

In certain instances, wherein the cell is a eukaryotic cell, the cell may be a cell typically associated with kidney tissue such as, but not limited to kidney parietal cells, kidney glomerulus podocyte cells, kidney proximal tubule brush border cells, loop of Henle thin segment cells, kidney distal tubule cells, kidney collecting duct cell, derivatives of these, and/or combinations of these.

In certain instances, wherein the cell is a eukaryotic cell, the cell may be a cell typically associated with ducts, such as, but not limited to nonstriated duct cells (of sweat gland, salivary gland, mammary gland, etc.), principal cells, intercalated cells, duct cells of the seminal vesicles, duct cells of the prostate gland, etc.), intestinal brush border cells, exocrine gland striated duct cells, gall bladder epithelial cells, ductulus efferens non-ciliated cells, epididymal principal cells, epididymal basal cells, derivatives of these, and/or combinations of these.

In some instances, wherein the cell is a eukaryotic cell, the cell may be a cell typically classified as a stromal cell such as, but not limited to adipocyte cells, lipoblast cells, fibroblast cells, fibrocyte cells, adventitial reticular cells, endothelial cells, undifferentiated mesenchymal cells, epithelial cells including squamous epithelial cells, cuboid epithelial cells, columnar epithelial cells, keratinocyte cells, melanocyte cells, Langerhans cells, pericyte cells, limbal stem cells, derivatives of these and/or combinations of these.

In certain instances, wherein the cell is a eukaryotic cell, the cell may be typically associated as a cell of the eye, such as, but not limited to rod cells, cone cells, retinal bipolar cells, retinal ganglion cells, amacrine cells, horizontal cells of the eye, iris pigmented epithelial cells, derivatives of these and/or combinations of these.

In certain instances, wherein the cell is a eukaryotic cell, the cell may typically be associated as a cell of the nose, such as, but not limited to microvilli equipped epithelial cells, basal cells of the nose, olfactory receptor cells, olfactory epithelial cells, derivatives of these and/or combinations of these.

In certain instances, wherein the cell is a eukaryotic cell, the cell may typically be associated as a cell of the eye, such as, but not limited to cochlear hair cells (inner hair cells, outer hair cells), Hansen cells, Boettcher cells, Claudius cells, pillar cells, derivatives of these and/or combinations of these.

In certain instances, wherein the cell is a eukaryotic cell, the cell may be a cell typically classified as a lung cell such as, but not limited to pneumocytes (e.g., type I pneumocytes, and type II pneumocytes), clara cells, goblet cells, derivatives of these and/or combinations of these.

In certain instances, wherein the cell is a eukaryotic cell, the cell may typically be classified as a cell of the musculoskeletal system such as, but not limited to cardiac myocyte cells, striated myocyte cells, smooth myocyte cells, myoblast cells, osteoblast cells, osteoclast cells, osteocyte cells, synoviocyte cells, chondroblast cells, chondrocyte cells, derivatives of these and/or combinations of these.

In certain instances, wherein the cell is a eukaryotic cell, the cell may be typically classified as a cell of the digestive system such as, but not limited to parietal cells, zymogenic cells, argentaffin cells of the duodenum, islets of Langerhans cells (alpha, beta, and delta), hepatocyte cells, kupfer cells, gastrin cells, enterochromaffin-like cells, gastric chief cells, foveolar cells, paneth cells, enterocyte cells, microfold cells, hepatic stellate cells, cholecystocyte cells, derivatives of these and/or combinations of these.

In certain instances, wherein the cell is a eukaryotic cell, the cell may be typically classified as a cell of the reproductive system such as, but not limited to oocytes, spermatozoa, Leydig cells, embryonic stem cells, amniocytes, blastocyst cells, morula cells, zygotes, epididymal principal cells, epididymal basal cells, derivatives of these and/or combinations of these.

In certain instances, wherein the cell is a eukaryotic cell, the cell may be typically classified as an endocrine cell such as, but not limited to somatotropic cells, mammotropic cells, gonadotropic cells, thyrotropic cells, corticotrophic cells, parafollicular cells, follicular cells, pancreatic stellate cells, pancreatic alpha cells, pancreatic beta cells, pancreatic delta cells, pancreatic polypeptide cells, pancreatic duct cells, adrenal cells, chromaffin cells, derivatives of these and/or combinations of these. In some cases, a cell can be a cell capable of secreting insulin. In some cases, a cell can be an islet cell.

In certain instances, the cell may be a blood stem cell such as a hematopoietic stem cell, a mesenchymal stem cell, a neural stem cell, an epithelial stem cell, a skin stem cell, a very small embryonic-like stem cell (VSELs), a peripheral blood stem cell (blastomeres), pluripotent stem cell, an epithelial cell, an endothelial cell, a fat cell, a red blood cell, a white blood cell, a platelet cell, a nerve cell, a skeletal cell, a cartilage cell, derivatives of these and/or combinations of these.

In certain cases, a cell can comprise a stem cells such as, but not limited to induced pluripotent stem cells (IPSC), totipotent stem cells, pluripotent stem cells, embryonic stem cells, multipotent stem cells, oligopotent stem cells, unipotent stem cells, hematopoietic stem cells, embryonic stem cells, stem cells derived from umbilical cord, derivatives of these and/or combinations of these.

In certain instances, cellular material may comprise Wharton's Jelly, a placenta, an umbilical cord, derivatives of these and/or combinations of these.

In certain instances, the cells listed in the preceding paragraphs is not exclusive. Certain cell types may additionally be classified by different names depending on tissue location. Certain cell types may be associated with multiple tissues and classifications.

In some cases, cells can be produced naturally or cultured. In some cases, spray drying (to form microencapsulated particles) can increase stabilization of a biological compound such as cellular material, proteins, peptides, monoclonal antibodies, enzymes, vaccines, or any combination thereof. In some cases, cellular material can be microencapsulated to preserve the cellular material for an increased amount of time as compared to a cellular material that is not microencapsulated. For example, a microencapsulated cellular material can be preserved for: 1 day, 2 days, 3 days, 4 days, 5 days, 6 days, 1 week, 2 weeks, 3 weeks, 4 weeks, 5 weeks, 1 month, 2 months, 3 months, 4 months, 5 months, 6 months, 7 months, 8 months, 9 months, 10 months, 11 months, 1 year, 2 years or longer as compared to a cellular material not microencapsulated. In some cases, the preservation of a cellular material can comprise maintaining cellular viability and/or maintaining biological activity of a cellular material.

In some embodiments, cellular material (e.g., a cellular component) can comprise a platelet-rich plasma (PRP), an exosome, or any combination thereof. In some instances, a cellular material can comprise a nucleic acid (e.g., DNA and/or RNA), a protein, a lipid, a carbohydrate, or any combination thereof. In some instances, a cellular material can comprise neuritin. In some cases, neuitin can comprise SEQ ID NO: 1. In some cases, neuritin can comprise a protein with at least about: 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence homology to SEQ ID NO: 1. In some cases, neutitin can comprise a protein with at least about: 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% percent length of SEQ ID NO: 1. In some cases, neuritin can comprise MGLKLNGRYISLILAVQIAYLVQA VRAAGKCDAVFKGFSDCLLKLGDSMANYPQGL DDKTNIKTVCTYWEDFHSCTVTALTDCQEGAKDMWDKLRKESKNLNIQGSLFELCG SGNGAAGSLLPAFPVLLVSLSAALATWLSF (SEQ ID NO: 1). In some cases, the cellular material herein can comprise a polynucleotide encoding neuritin. In some cases, the polynucleotide encoding neuritin can comprise a sequence with at least about: 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to ATGGGCCTGAAACTGAACGGCCGCTATATTAGCCTGATTCTGGCGGTGCAGATTG CGTATCTGGTGCAGGCGGTGCGCGCGGCGGGCAAATGCGATGCGGTGTTTAAAG GCTTTAGCGATTGCCTGCTGAAACTGGGCGATAGCATGGCGAACTATCCGCAGG GCCTGGATGATAAAACCAACATTAAAACCGTGTGCACCTATTGGGAAGATTTTCA TAGCTGCACCGTGACCGCGCTGACCGATTGCCAGGAAGGCGCGAAAGATATGTG GGATAAACTGCGCAAAGAAAGCAAAAACCTGAACATTCAGGGCAGCCTGTTTGA ACTGTGCGGCAGCGGCAACGGCGCGGCGGGCAGCCTGCTGCCGGCGTTTCCGGT GCTGCTGGTGAGCCTGAGCGCGGCGCTGGCGACCTGGCTGAGCTTT (SEQ ID NO: 2). In some cases, the polynucleotide encoding neuritin can comprise a sequence with at least about: 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% percent length of SEQ ID NO: 2. In some cases, the polynucleotide encoding neuritin can comprise a sequence with at least about: 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% to identity AUGGGCCUGAAACUGAACGGCCGCUAUAUUAGCCUGAUUCUGGCGGUGCAGA UUGCGUAUCUGGUGCAGGCGGUGCGCGCGGCGGGCAAAUGCGAUGCGGUGUU UAAAGGCUUUAGCGAUUGCCUGCUGAAACUGGGCGAUAGCAUGGCGAACUAU CCGCAGGGCCUGGAUGAUAAAACCAACAUUAAAACCGUGUGCACCUAUUGGGA AGAUUUUCAUAGCUGCACCGUGACCGCGCUGACCGAUUGCCAGGAAGGCGCGA AAGAUAUGUGGGAUAAACUGCGCAAAGAAAGCAAAAACCUGAACAUUCAGGG CAGCCUGUUUGAACUGUGCGGCAGCGGCAACGGCGCGGCGGGCAGCCUGCUGC CGGCGUUUCCGGUGCUGCUGGUGAGCCUGAGCGCGGCGCUGGCGACCUGGCUG AGCUUU (SEQ ID NO: 3). In some cases, the polynucleotide encoding neuritin can comprise a sequence with at least about: 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% percent length of SEQ ID NO: 3. In some cases, a microencapsulated particle herein can comprise the polynucleotide of SEQ ID NO: 2 and/or SEQ ID NO: 3.

In some cases, a composition herein can comprise a nucleic acid such as DNA or RNA encoding SEQ ID NO: 1. In some cases, neuritin or a cellular material can be administered concurrently or consecutively with a therapeutically effective amount of a bronchodilator, a corticosteroid, an asthma controller medication, or any combination thereof. In some cases, the bronchodilator can be, for example, albuterol, a long acting bronchodilator, such as, salmeterol, or formoterol. In some cases, the corticosteroid can comprise budesonide, fluticasone, beclometasone, prednisone, ciclesonide, or any combination thereof. In some cases, an asthma controller medication can comprise benralizumab, montelukast, omalizumab, or a combination thereof. In some cases, the bronchodilator, the corticosteroid, or the asthma controller medication can be administered by any method described herein, such as by inhalation, by intravenous administration, or by oral administration. In some cases, the dose of the bronchodilator, the corticosteroid, or the asthma controller medication can range from about: 0.0001 mg to 1000 mg, 0.001 mg to 100 mg, 0.01 mg to 10 mg, or 0.1 mg to 1 mg.

In some cases, a dosage form can comprise a mixture of cell types, for example a dosage form can comprise about 1, 2, 3, 4, 5, 6, 7, 8, 9, 10 or more cell types. In some cases, a dosage form can comprise a mixture of cellular material, for example a dosage form can comprise about 1, 2, 3, 4, 5, 6, 7, 8, 9, 10 or more cellular materials (e.g., cellular components). In some cases, a dosage form can comprise a mixture of cell types and a mixture of cellular material.

In some embodiments, an active ingredient described herein can comprise a derivative of an active ingredient described herein. In some cases, a derivative of a cellular component disclosed herein, can refer to a biological or chemical substance related structurally to a compound or biological material disclosed herein. A derivative can be made from a structurally related parent compound in one or more steps. In some cases, the general physical and chemical properties of a derivative can be similar to a parent compound. In some cases, a derivative of a cell can refer to the derivative cell having similar phenotypic traits to the cell it is a derivative of.

In some embodiments, an active pharmaceutical ingredient or salt thereof can be formulated as a powder. For example, a cellular material disclosed herein can be formulated as a powder using the methods described herein.

In some embodiments, the active pharmaceutical ingredients can comprise phosphodiesterase inhibitors or pharmaceutically acceptable salts thereof. In some instances, the phosphodiesterase inhibitors can be phosphodiesterase type 5 inhibitors (PDE5 inhibitors). In some instances, the phosphodiesterase type 5 inhibitors can include Sildenafil Citrate (Viagra), Tadalafil (Cialis) Avanafil (Stendra), and Vardenafil Hydrochloride (Levitra). In some cases, a PDE-V inhibitor can comprise sildenafil, tadalafil, avanafil, vardenafil, an ester thereof, a salt thereof, or any combination thereof. In some cases, a PDE-V inhibitor can comprise mirodenafil, udenafil, lodenafil, zaprinast, icariin, an ester of any of these, a pharmaceutically acceptable salt of any of these, or any combination thereof. In some cases, a PDE-V inhibitor can comprise lodenafil carbonate. In some cases, a phosphodiesterase inhibitor can comprise a selective phosphodiesterase inhibitor, a nonselective phosphodiesterase inhibitor, a PDE-I selective inhibitor, a PDE-II selective inhibitor (e.g. EHNA (erythro-9-(2-hydroxy-3-nonyl) adenine)), a PDE-III selective inhibitor, a PDE-IV selective inhibitor, a PDE-V selective inhibitor, a PDE-VI selective inhibitor, a PDE-VII selective inhibitor, a PDE-IX selective inhibitor, a PDE-X selective inhibitor, a PDE-XI selective inhibitor, a pharmaceutically acceptable salt of any of these, or any combination thereof. In some cases, an active pharmaceutical ingredient can comprise oxindole, inamrinone, anagrelide, cilostazol, mesembrenone, rolipram, ibudilast, roflumilast, apremilast, cisaborole, sildenafil, tadalafil, vardenafil, udenafil, avanafil, dipyridamole, quinazoline, paraxanthine, papaverine, a pharmaceutically acceptable salt of any of these, an ester of any of these, or any combination thereof. In some cases, a PDE5 inhibitor or a salt thereof such as sildenafil or a salt thereof can be administered in a composition comprising a cellular material described herein.

In some embodiments, active pharmaceutical ingredients or salts thereof can comprise an antibiotic, an antiviral, an antiparasitic, a diuretic, a blood pressure medication, a phosphodiesterase inhibitor, a pharmaceutically acceptable salt of any of these, or any combination thereof. In some cases, an active pharmaceutical ingredient can comprise a beta-blocker, an ACE inhibitor, an angiotensin II receptor blocker, a calcium channel blocker, an alpha blocker, a cancer chemotherapeutic, a steroid, an immunomodulator, a pharmaceutically acceptable salt of any of these, or any combination thereof. In some cases, an antibiotic can comprise a penicillin, a cephalosporin, a tetracycline, an aminoglycoside, a macrolide, clindamycin, a sulfonamide, a trimethoprim, a metronidazole, a quinolone, or a nitrofurantoin. An antiviral can comprise an acyclovir, peramivir, zanamivir, oseltamivir phosphate, remdesivir, balozavir marboxil, a salt of any of these or any combination thereof.

In some embodiments, the composition can further comprise: another set of active pharmaceutical ingredients or salts thereof. For example, a second, third, or fourth different set of active pharmaceutical ingredients. In some instances, the additional pharmaceutical ingredients or salts thereof can be administered in concurrently or consecutively to enhance the efficacy of the first set of active pharmaceutical ingredients or salts.

In some embodiments, the first set of active pharmaceutical components or a first active pharmaceutical component can be administered concurrently or consecutively with a second different set of active pharmaceutical components or a second pharmaceutical component. In some cases, the pharmaceutical components can comprise nitrates, nitric oxide, nitric oxide generating components, nitrite salts, nitrate salts, sodium nitrates, potassium nitrates, vitamin C, ascorbic acid, L-arginine, L-citrulline, vitamin B12, magnesium ascorbate, sodium ascorbate, potassium ascorbate, antihypertensive agents, diuretics, salts thereof, or any combination thereof. In some cases, the pharmaceutical ingredients can comprise beta blockers (B-blockers), calcium blockers, angiotensin converting enzyme inhibitors, angiotensin receptor blockers, Nebivolol, CYP3A4 inhibitors, ketoconazole (Nizoral), itraconazole (Sporanox), erythromycin, saquinavir, clarithromycin, HIV protease inhibitors, alpha-adrenergic blocking agents (a-blockers), salts thereof, or any combination thereof.

In some embodiments, a pharmaceutical composition can have metabolites that can be pharmacologically active, retaining, at least partially, the potency of the parent drug or the parent pharmaceutical component.

### Excipients and Other Non-Active Ingredients

In some embodiments, the pharmaceutical composition comprises pharmaceutically acceptable excipients. In some instances, an excipient can comprise a substance formulated alongside the active ingredient of a medication, included for the purpose of long-term stabilization, bulking up solid formulations that contain potent active ingredients in small amounts, and/or to confer a therapeutic enhancement on the active ingredient(s) in the final dosage form. Excipients may facilitate drug absorption, reduce viscosity, or enhance solubility. Excipients may also facilitate the handling of the active ingredients, improve in vitro stability, and/or extend pharmaceutical product shelf life. Excipient selection may vary with the route of administration for drug delivery, the unit dose, as well as the active ingredients comprising the composition.

In some embodiments, an excipient can comprise ab anhydrous calcium phosphate, a dihydrate calcium phosphate, a hydroxypropyl methylcellulose, a croscarmellose sodium, a GMO-free croscarmellose sodium, a carbomers, a magnesium aluminometasilicate, a mannitol, a povidone (PVP), a crospovidone, a sorbitol, a dimethicone, a sodium stearyl fumarate, a sodium starch glycollate, a hydroxypropylcellulose, a native corn starch, a modified corn starch, a carrageenan, an alginates, a silicon dioxide, a microcrystalline cellulose, a carboxymethylcellulose sodium, an alginates, a carboxymethylcellulose (CMC), a sodium carboxymethylcellulose (Na CMC), a carbomers, a natural gums, a sorbitol, a maltitol, a glucose syrup, a silicon, a carbomer, a fatty alcohol, an alcohol, a carbohydrate, a petrolatum derivative, a butter, a wax, DMSO Procipient®, am ester, a fatty acid, an oil-in-water (O/W) emulsifier, a water-in-oil (W/O) emulsifier, a silica, a fumed silica, a polysorbate, an isopropyl myristate, a cellulosic derivate, a xanthan gum, a propylenglycol, a noveon AA-1 polycarbophyl, a dimethyl isosorbate, a polysilicone elastomer 1100, a polysilicone elastomer 1148P, a preservative, a flavor, a color, a functional coating, an aesthetic coating, a pharmaceutically acceptable salt of any of these, or any combination thereof.

In some cases, a pharmaceutically acceptable excipient can comprise acacia, acesulfame potassium, acetic acid, glacial, acetone, acetyl tributyl citrate, acetyl triethyl citrate, agar, albumin, alcohol, alginic acid, aliphatic polyesters, alitame, almond oil, alpha tocopherol, aluminum hydroxide adjuvant, aluminum oxide, aluminum phosphate adjuvant, aluminum stearate, ammonia solution, ammonium alginate, ascorbic acid, ascorbyl palmitate, aspartame, attapulgite, bentonite, benzalkonium chloride, benzethonium chloride, benzoic acid, benzyl alcohol, benzyl benzoate, boric acid, bronopol, butylated hydroxyanisole, butylated hydroxytoluene, butylparaben, calcium alginate, calcium carbonate, calcium phosphate, dibasic anhydrous, calcium phosphate, dibasic dihydrate, calcium phosphate, tribasic, calcium stearate, calcium sulfate, canola oil, carbomer, carbon dioxide, carboxymethylcellulose calcium, carboxymethylcellulose sodium, carrageenan, castor oil, castor oil, hydrogenated, cellulose (e.g. microcrystalline, powdered, silicified microcrystalline, acetate, acetate phthalate) ceratonia, cetostearyl alcohol, cetrimide, cetyl alcohol, cetylpyridinium chloride, chitosan, chlorhexidine, chlorobutanol, chlorocresol, chlorodifluoroethane, chlorofluorocarbons, chloroxylenol, cholesterol, citric acid monohydrate, colloidal silicon dioxide, coloring agents, copovidone, corn oil, cottonseed oil, cresol, croscarmellose sodium, crospovidone, cyclodextrins, cyclomethicone, denatonium benzoate, dextrates, dextrin, dextrose, dibutyl phthalate, dibutyl sebacate, diethanolamine, diethyl phthalate, difluoroethane, dimethicone, dimethyl ether, dimethyl phthalate, dimethyl sulfoxide, dimethylacetamide, disodium edetate, docusate sodium, edetic acid, erythorbic acid, erythritol, ethyl acetate, ethyl lactate, ethyl maltol, ethyl oleate, ethyl vanillin, ethylcellulose, ethylene glycol palmitostearate, ethylene vinyl acetate, ethylparaben, fructose, fumaric acid, gelatin, glucose, glycerin, glyceryl behenate, glyceryl monooleate, glyceryl monostearate, glyceryl palmitostearate, glycofurol, guar gum, hectorite, heptafluoropropane, hexetidine, hydrocarbons, hydrochloric acid, hydroxyethyl cellulose, hydroxyethylmethyl cellulose, hydroxypropyl cellulose, hydroxypropyl cellulose, low-substituted, hydroxypropyl starch, hypromellose, hypromellose acetate succinate, hypromellose phthalate, imidurea, inulin, iron oxides, isomalt, isopropyl alcohol, isopropyl myristate, isopropyl palmitate, kaolin, lactic acid, lactitol, lactose, anhydrous, lactose, monohydrate, lactose, spray-dried, lanolin, lanolin alcohols, lanolin, hydrous, lauric acid, lecithin, leucine, linoleic acid, macrogol hydroxystearate, magnesium aluminum silicate, magnesium carbonate, magnesium oxide, magnesium silicate, magnesium stearate, magnesium trisilicate, malic acid, maltitol, maltitol solution, maltodextrin, maltol, maltose, mannitol, medium-chain triglycerides, meglumine, menthol, methylcellulose, methylparaben, mineral oil, mineral oil, light, mineral oil and lanolin alcohols, monoethanolamine, monosodium glutamate, monothioglycerol, myristic acid, neohesperidin dihydrochalcone, nitrogen, nitrous oxide, octyldodecanol, oleic acid, oleyl alcohol, olive oil, palmitic acid, paraffin, peanut oil, pectin, petrolatum, petrolatum and lanolin alcohols, phenol, phenoxyethanol, phenylethyl alcohol, phenylmercuric acetate, phenylmercuric borate, phenylmercuric nitrate, phosphoric acid, polacrilin potassium, poloxamer, polycarbophil, polydextrose, polyethylene glycol, polyethylene oxide, polymethacrylates, poly(methyl vinyl ether/maleic anhydride), polyoxyethylene alkyl ethers, polyoxyethylene castor oil derivatives, polyoxyethylene sorbitan fatty acid esters, polyoxyethylene stearates, polyvinyl acetate phthalate, polyvinyl alcohol, potassium alginate, potassium benzoate, potassium bicarbonate, potassium chloride, potassium citrate, potassium hydroxide, potassium metabisulfite, potassium sorbate, povidone, propionic acid, propyl gallate, propylene carbonate, propylene glycol, propylene glycol alginate, propylparaben, 2-pyrrolidone, raffinose, saccharin, saccharin sodium, saponite, sesame oil, shellac, simethicone, sodium acetate, sodium alginate, sodium ascorbate, sodium benzoate, sodium bicarbonate, sodium borate, sodium chloride, sodium citrate dihydrate, sodium cyclamate, sodium hyaluronate, sodium hydroxide, sodium lactate, sodium lauryl sulfate, sodium metabisulfite, sodium phosphate, dibasic, sodium phosphate, monobasic, sodium propionate, sodium starch glycolate, sodium stearyl fumarate, sodium sulfite, sorbic acid, sorbitan esters (sorbitan fatty acid esters), sorbitol, soybean oil, starch, starch (e.g. pregelatinized, sterilizable maize), stearic acid, stearyl alcohol, sucralose, sucrose, sugar, compressible, sugar, confectioner's, sugar spheres, sulfobutylether b-cyclodextrin, sulfuric acid, sunflower oil, suppository bases, hard fat, talc, tartaric acid, tetrafluoroethane, thaumatin, thimerosal, thymol, titanium dioxide, tragacanth, trehalose, triacetin, tributyl citrate, triethanolamine, triethyl citrate, vanillin, vegetable oil, hydrogenated, water, wax, anionic emulsifying, wax (e.g. carnauba, cetyl esters, microcrystalline, nonionic emulsifying, white, yellow), xanthan gum, xylitol, zein, zinc acetate, zinc stearate, or any combination thereof.

In some embodiments, a pharmaceutically acceptable excipient can comprise a carbohydrate, an alginate, povidone, a carbomer, a flavor, a natural gum, a silicone, an alcohol, a butter, a wax, a fatty acid, a preservative, a pharmaceutically acceptable salt of any of these, or any combination thereof. In some instances, a pharmaceutically acceptable excipient can comprise a carbohydrate. In some instances, the carbohydrate can comprise a lactose, a microcrystalline cellulose, a cellulose, a mannitol, a sorbitol, a starch, a starch glycolate, a hydroxypropyl methylcellulose, a hydroxypropyl methylcellulose acetate succinate, a cyclodextrin, a maltodextrin, a croscarmellose sodium, a corn starch, a carrageenan, a sorbitol, a maltitol, a glucose, a pharmaceutically acceptable salt of any of these, or any combination thereof. In some instances, a pharmaceutically acceptable excipient can comprise lactose. In some instances, lactose can comprise a milled lactose, a sieved lactose, a micronized lactose, a spray dried lactose, an anhydrous lactose, a monohydrate lactose, or a combination thereof.

In some embodiments, blending of a cellular material (e.g., at least partially encapsulated cells, at least partially encapsulated one or more cellular components, or both) can utilize a V-type blender (for powders) to distribute the cellular material uniformly into the excipient carrier. In some cases, the cellular material can be admixed with an excipient in a substantially homogenous mixture. The V-Blenders can be a blender manufactured by Patterson Kelly/PK Blender, Gemco or Ross blenders.

In some embodiments, the active ingredient or pharmaceutically acceptable salt thereof can be contained at least in part within an excipient. In some instances, the active ingredient or pharmaceutically acceptable salt thereof can be contained at least in part in an excipient. In some instances, the active ingredient can be contained within a pore of an excipient. The “pore” of the excipient can refer to excipient particles that have been engineered to have open or closed pore structures. Porous excipient particles may be carriers of pharmaceutically active ingredients. Porous excipient particles may have a large surface area, stable structure, adjustable pore sizes, tunable dissolution, diffusion, or distribution, and well-defined surface properties. Porous excipient particles may facilitate sustained-release unit doses.

In some embodiments, in addition to the active pharmaceutical ingredients or salts thereof, the compositions can further comprise inactive ingredients selected from the group consisting of microcrystalline cellulose, anhydrous dibasic calcium phosphate, croscarmellose sodium, magnesium stearate, hypromellose, titanium dioxide, lactose, triacetin, mannitol, xylitol, sorbitol, sugar alcohols, cellulose, cellulose esters, cellulose ethers, modified celluloses, starch, modified starches, polysaccharides, oligosaccharides, disaccharides, saccharides, gelatin, polyvinylpyrrolidone, polyethylene glycol, binders, flavorants, colorants, FD & C Blue #2 aluminum lake, magnesium stearate, antiadherent agents, stearate salts, sweeteners, silica, lubricants, or any combination thereof.

### Salts

In some embodiments, the pharmaceutical composition comprising the salt of the pharmaceutically active ingredient, wherein the salt comprises an organic salt, an inorganic salt, or any combination thereof. In some cases, an organic salt can comprise a phosphinate (e.g. sodium hypophosphite), a hydrazinium salt, a urate, a diazonium salt, an oxalate salt, a tartrate, a choline chloride. An example of an inorganic salt can be sodium chloride, calcium chloride, magnesium chloride, sodium bicarbonate, potassium chloride, sodium sulfate, calcium carbonate, calcium phosphate, or any combination thereof.

In some embodiments, the pharmaceutical composition comprising the salt of the pharmaceutically active ingredient, wherein the salt comprises an HCl salt, an ascorbic acid salt, a mandelic acid salt, an aspartic acid salt, a carbonic acid salt, a citric acid salt, a formic acid salt, a glutamic acid salt, a lactic acid salt, a lauric acid salt, a maleic acid salt, a palmitic acid salt, a phosphoric acid salt, or any combination thereof.

In some embodiments, the pharmaceutically acceptable salts include, but are not limited to, metal salts such as sodium salt, potassium salt, cesium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like; inorganic acid salts such as hydrochloride, hydrobromide, phosphate, sulphate and the like; organic acid salts such as citrate, lactate, tartrate, maleate, fumarate, mandelate, acetate, dichloroacetate, trifluoroacetate, oxalate, formate and the like; sulfonates such as methanesulfonate, benzenesulfonate, p-toluenesulfonate and the like; and amino acid salts.

### Method of Making the Pharmaceutical Composition

In some cases, methods of making a pharmaceutical composition can comprise creating particles by the methods described herein. In some cases, particles can comprise an excipient (e.g. a pharmaceutically acceptable excipient), an active ingredient, or both. In some instances, a method of making a powdery pharmaceutical composition, can comprise mixing, in a mixer, particles of a pharmaceutically acceptable excipient; and particles comprising an active ingredient at least partially encapsulated in a coating material and wherein the particles at least partially encapsulated in the coating material are spray dried. In some instances, a method of making a powdery pharmaceutical composition, can comprise contacting particles of a pharmaceutically acceptable excipient and particles comprising a cellular material at least partially encapsulated in a coating material, wherein the particles at least partially encapsulated in the coating material are spray dried.

In some cases, a composition can comprise a mixture of particles described herein. In some instances, at least a portion of the particles of the pharmaceutically acceptable excipient have a particle diameter ranging from about 50 μm to about 200 μm, as measured by a particle size analyzer using laser diffraction; at least a portion of the particles comprising the active ingredient, or the pharmaceutically acceptable salt thereof encapsulated in the coating material can have a particle diameter ranging from about: 1 μm to about 100 μm, 10 μm to about 50 μm, or from about 1 μm to about 200 μm, as measured by a particle analyzer using laser diffraction.

In some embodiments, the method of making a composition can comprise formulating the particles described herein into a capsule-in-capsule composition (e.g., a pharmaceutical composition). In some cases, particles can comprise an excipient, an active ingredient, or both. In some cases, particles can comprise a carrier, an active ingredient, or both. In some cases, particles can comprise a diluent, an active ingredient, or both. In some instances, a capsule-in-capsule formulation can be in unit dose form. In some cases, the formulation can comprise particles comprising a cellular material, or a pharmaceutically acceptable salt thereof. In some instances, the particles can be at least partially encapsulated by a coating material. In some instances, the particles at least partially encapsulated by the coating material can be spray dried. In some cases, the particles can be at least partially surrounded by a first capsule, a second capsule, or both. In some instances, the first capsule can be surrounded by a second capsule to create a capsule-in-capsule, capsule. In some cases, a capsule can comprise a capsule coating. In some cases, a capsule coating can at least partially control capsule ingredient release.

In some embodiments, a final product can be a capsule-in-capsule. In some cases, the final product can be a capsule (e.g., a second capsule) that surrounds an active ingredient (e.g., a cellular material) and separately an inner capsule (e.g., the first capsule), which can contain its own active ingredient. In some instances, a capsule can contain more than one active ingredient. In some instances, a capsule can contain more than one inner capsule. For example, a capsule can comprise 1, 2, 3, 4, 5, 6, 7, 8, 9, 10 or more inner capsules. In some cases, an inner capsule can comprise a capsule. For example, an outer capsule can surround a first inner capsule and the first inner capsule can surround a second inner capsule.

The process described herein can include the following manufacturing stages. The active ingredient of the first capsule and the second capsule can be microencapsulated and spray dried using the methods described herein. In some instances, the active ingredients can be independently blended with an excipient. In some cases, the active ingredients may not be blended with an excipient. In some cases, the active ingredient of the first capsule can then be added to the first capsule and the first capsule can be banded using the methods described herein. In some cases, after the first capsule is banded, a capsule coating (e.g. an enteric, pH dependent, time release, or combination release) can be applied to the first capsule. In some cases, the active ingredient of the second capsule can then be added to the second capsule and the first capsule can be placed into the second capsule. The second capsule can be banded, and a capsule coating can be applied to the second capsule.

In some cases, a composition can comprise a mixture of particles described herein. In some instances, at least a portion of an excipient and at least a portion of the particles comprising an active ingredient, can comprise a mixture or a formulation.

In some embodiments, a method of making the powdery pharmaceutical composition can comprise the following steps: microencapsulation of active pharmaceutical ingredient; spray drying, atomization and dry powder collection, blending of active pharmaceutical ingredient with excipient; and encapsulation in a capsule or storage in a container.

### Microencapsulation

In some embodiments, encapsulation of an active ingredient such as a cellular material can comprise microencapsulation.

Microencapsulation can be a process in which a microcapsule can be created as a small sphere or multi-sphere with a core and a matrix wall around it. The pharmaceutical ingredient inside the microcapsule can be called a fill. In some cases, a fill can be a liquid, an oil, a solid, or any combination thereof. In some cases, a fill can be a cellular material, such as a cell. The wall around the fill (“or core”) can be referred to as a shell, a coating, or a membrane. In some cases, a microcapsule can comprise more than one shell. For example, a microcapsule can comprise 2, 3, 4, 5, 6, 7, 8, 9, or more shells. In some cases, a small size of a microencapsulated particle can provide an active ingredient a large surface area. In some cases, the small size of a microencapsulated particle can provide an active ingredient a large surface area to be available for absorption, release, transfer, or any combination thereof.

In some cases, a microencapsulated shell can comprise a coating to allow delivery to another part of the body or to the area where the composition was originally delivered. For example, a microencapsulated shell can comprise an enteric coating, a time release coating, a pH dependent coating, a delayed release coating, an extended-release coating, or a combination thereof. In some cases, the microencapsulated shell can be thicker to deliver the cell further into the body. In some cases, the microencapsulated shell can be a mixture of thicker and thinner shell sizes to have a controlled release. In some cases, the microencapsulated shell can comprise a controlled released coating or a mixture of controlled release coatings to provide dosing throughout different parts of the body. For example, microencapsulated cells can be configured to release in the small intestine and in the large intestine.

In some embodiments, a microencapsulated particle can be configured (for example, with one or more layers of an enteric coating) to at least partially release an active ingredient in: the mouth, the esophagus, the small intestine, the duodenum, the jejunum, the ileum, the cecum, the colon, the ascending colon, the traverse colon, the descending colon, the sigmoid colon, the rectum, the anus, or any combination thereof.

In some cases, microencapsulation can at least partially prevent inhalation of an active ingredient comprising the form of an unencapsulated crystal. In some instances, unencapsulated crystals can cause irritation of the respiratory tract of a subject during inhalation. The irritation can be caused by crystal geometry and structure. For example, a crystal can have sharp angles and edges that can cause irritation, damage or both of the respiratory tract during inhalation. In some instances, crystal geometry and structure can be controlled by the spray drying process. Microencapsulation can generate crystals with amorphous structure. In some instances, an amorphous crystal can lack sharp edges and angles. In some cases, an amorphous crystal can have a rounded edge. In some instances, an amorphous crystal may have increased bioavailability.

In some instances, a pharmaceutical composition in oil formulation can be microencapsulated with compatible diluents to protect the oil from oxidation and provide a longer shelf life than the unprotected pharmaceutical composition. The diluents can be aqueous, or solvent based and use animal or plant materials. In some cases, the diluent can comprise alcohols: e.g., ethanol, butanol, 2-ethylhexanol, isobutanol, isopropanol, methanol, propanol, propylene glycol; ketones: e.g., acetone, methyl ethyl ketone, methyl isobutyl ketone, methyl isopropyl ketone, mesityl oxide, trichloroethylene; halogenated solvents: e.g., ethylene bromide, chloroform, ethylene chloride, dichloromethane, tetrachloroethylene, carbon tetrachloride; amides: e.g., dimethylformamide; ethers: e.g., 1,4-dioxane, butyl ether, ethyl ether, di-isopropyl ether, tetrahydrofuran, tert-butyl methyl ether; sulfur containing solvent: e.g., dimethyl sulfoxide; amines: e.g., pyridine; nitriles: e.g., acetonitrile; esters: e.g., ethyl acetate; aliphatic hydrocarbons: e.g., cyclohexane hexane; aromatic hydrocarbons: e.g., toluene xylene; water or any combinations thereof. In some cases, the diluent can comprise benzene, carbon tetrachloride, 1,2-dichloroethane, 1,1-dichloroethene, 1,1,1-trichloroethane, acetonitrile, chlorobenzene, chloroform, cyclohexane, 1,2-dichloroethene, dichloromethane, 1,2-dimethoxyethane, N,N-dimethylacetamide, N,N-dimethylformamide, 1,4-dioxane, 2-ethoxyethanol, ethylene glycol, formamide, hexane, methanol, 2-methoxyethanol, methylbutylketone, methylcyclohexane, n-methylpyrrolidone, nitromethane, pyridine, sulfolane, tetralin, toluene, 1,1,2-trichloroethylene, xylene or any combinations thereof.

In some embodiments, an inhaled powder can adapt well with small to large proteins and can be readily accepted in the body due to the permeable, large absorptive surface area in the alveolar region in the lungs. After the microencapsulation process, the suspension can be spray dried to create the dry powder finished product.

In some embodiments, a particle that has previously been microencapsulated can be microencapsulated with another coating. For example, an initial coating can be applied to cover the fill (e.g., the active ingredient) then a second coating can be applied to the first coating. In some cases, a third coating can be applied to the second coating. In some cases, a fourth coating can be applied to the third coating. In some cases, a fifth coating can be applied to a fourth coating. In some cases, a sixth coating can be applied to the fifth coating. In some cases, a seventh coating can be applied to a sixth coating. In some cases, an eighth coating can be applied to the seventh coating. In some cases, a ninth coating can be applied to the eighth coating. In some cases, additional coatings can be applied to the ninth coating.

In some embodiments, after each microencapsulation process, the suspension can be spray dried to create a dry powder product. In some embodiments, after a particle has been microencapsulated, an addition coating, for example, an enteric coating, or any coating described herein may be applied by a fluidized bed system.

Spray drying, atomization and dry powder collection

In some embodiments, a method of making the powdery pharmaceutical composition can comprise particles wherein at least a portion of the particles of the active ingredient or a pharmaceutically acceptable salt thereof can be made by a spray drying process.

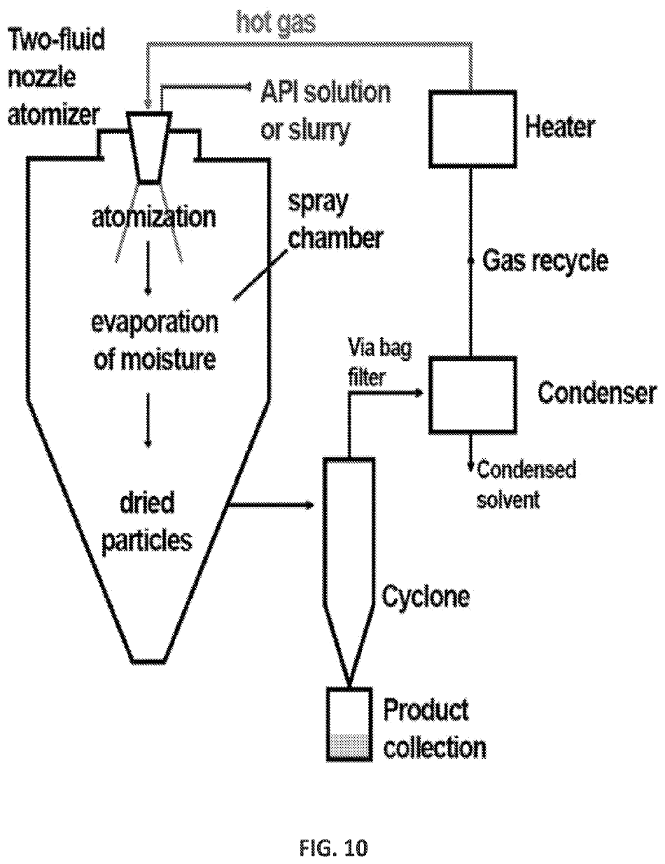

FIG. 10 illustrates a spray drying process. In some instances, a spray drying process can be broken down into three stages: 1) atomization, 2) drying and particle formation, and 3) recovery. In some cases, atomization can comprise a liquid feed stock that which can be atomized into droplets by means of a nozzle or rotary atomizer. In some cases, a nozzle can use air pressure or compressed gas (e.g., air, CO2, Nitrogen) to atomize the liquid feed stock while rotary atomizers can employ an atomizer wheel rotating at high speed. In some cases, the drying and particle formation can comprise, guiding by a gas disperser, a process gas (air, CO2 or Nitrogen) contacting the atomized feed to initiate evaporation. In some cases, as the liquid rapidly evaporates from the droplet surface, a solid particle can form and can fall to the bottom of the drying chamber. In some instances, the balance between temperature, flow rate and droplet size can control the drying process. In some cases, the recovery can comprise recovering the powder from the exhaust gas using a cyclone or a bag filter.

In some embodiments, the spray drying process can comprise: atomizing liquid droplets comprising the active ingredient or the pharmaceutically acceptable salt thereof, drying the droplets from particles, recovering the particles, or any combination thereof. In some cases, the liquid droplets can comprise an encapsulated active ingredient.

In some embodiments, the process can be a closed loop operation so that the solvents and nitrogen gas are not exhausted to the atmosphere. The closed-loop system can be configured to recycle (reheat and reuse and pass through a condenser) the gases/solvent outlet gases. The closed loop process recirculates the solvent vapor from the operation back into the system. The amount of solvent condensed out determines the drying and particle formation. Conversely, an open loop cycle can exhaust gases to the atmosphere in one pass and may be used for certain cellular material.

In some embodiments, spray drying may have advantages over lyophilization technology, which can be more costly and may require the cellular material to be mixed with DMSO, ethylene glycol and sucrose so that the freezing process does not damage the cellular structure. In some instances, the spray dry technology can allow for the thawing of the cells to be controlled. In some cases, the spray drying process can eliminate the need for harmful chemical agents.

In some embodiments, a spray drying manufacturing system can comprise a closed spray dryer container which receives the solution comprising a cellular material mixed with a suitable solvent (e.g., aqueous or solvent based). In some instances, the solution then enters the particle formation chamber which can be connected to an atomizer located at the top of the chamber. In some cases, the atomizer can use a gas, such as air. In some instances, the atomizer can be a two component or rotary nozzle type that distributes the solution into fine droplets controlled by the atomizer pressure. In some instances, the atomizer can be a rotary atomizer that employ an atomizer wheel rotating at high speed.

In some cases, a solvent can comprise a water, a saline, an alcohol, an ethanol, a dimethylformamide (DMF), a dimethyl sulfoxide (DMSO), a polar organic solvent, an organic solvent, or any combination thereof. In some cases, a solvent can comprise isopropyl alcohol. In some cases, a solvent can comprise a long chain alcohol such as cetyl alcohol or octyl alcohol. In some cases, a solvent can comprise a liposome, a sugary solvent, such as a sugar polyethylene glycol (PEG) like a glucose PEG, a galactose PEG, a mannose PEG, a trehalose PEG, or any combination thereof. In some cases, a solvent can comprise a glycerin, a glycol, or a combination thereof. In some cases, a solvent can comprise a grain alcohol, a longer chain alcohol, a butyl alcohol, or any combination thereof.

In some embodiments, an atomization gas can be an inert gas. In some cases, an atomization gas can be air. As used herein, “inert gas” can refer to a non-reactive gas, or a gas that does not undergo chemical reactions under a set of given conditions. Inert gases can be generally used to avoid unwanted chemical reactions degrading a sample, or to prevent bacterial growth. These undesirable chemical reactions can often be oxidation and hydrolysis reactions with the oxygen and moisture in air. The term “inert gas” can be context-dependent because several of the noble gases, which have been historically referred to as the inert gases, can be made to react under certain conditions. In some instances, inert gas can be nitrogen, carbon dioxide or any combination thereof. In some instances, the atomized droplets go through a hot gas drying chamber to produce uniform fine particles that maintain a tight particle size distribution following liquid evaporation. In some cases, the solid particle forms and falls to the bottom of the drying chamber. In some instances, the balance between temperature, flow rate, and droplet size can control the drying process. In some instances, the powder can be recovered from the exhaust gas using a cyclone or a bag filter.

The moisture level of the powder after spray drying can be below about 10%. In some embodiments, the moisture level can be below about 15%, about 14%, about 13%, about 12%, about 11%, about 10%, about 9%, about 8%, about 7%, about 6%, about 5%, about 4%, about 3%, about 2%, or about 1%.

Blending of Active Pharmaceutical Ingredient with Excipient

In some embodiments, a particle size can be validated by a Malvern particle analyzer prior to blending with an excipient carrier. In some instances, the active powder (e.g. the powdery pharmaceutical composition) can be blended with an excipient carrier (product in a Patterson Kelly (PK Blender) and the blended powder can be fed to a hopper.

### Encapsulation

The core active ingredient (e.g., a cellular material) can be microencapsulated with an amphipathic molecule that has both a polar end (‘hydrophilic”) and non-polar end (“hydrophobic”). In some cases, a hydrophilic end of an amphipathic molecule may interact with the core material In some cases, a hydrophobic end of an amphipathic molecule may interact with the core material. This hydrophilic and hydrophobic structure can enable the molecule to microencapsulate an active ingredient and form a microsphere. In some instances, the microencapsulated particle may have a hydrophilic exterior and a hydrophobic interior In some instances, the microencapsulated particle may have a hydrophobic exterior and a hydrophilic interior. The microencapsulation process can coat the active ingredient, which is the core, by the amphipathic encapsulating agent, which is the wall material, so that the active ingredient is at least partially surrounded by a wall of the amphipathic material. For example, hydroxypropyl methylcellulose acetate succinate (HPMCAS) can be an amphipathic molecule used to coat a cellular material. The microencapsulation blend can be a spray dried dispersion, that can be fed into a spray dry system to create a hard-outer coating on the microcapsules. In some instances, a microencapsulated cellular material can also contain nutrients for an encapsulated cell to maintain viability. In some cases, more than one coating can be applied to a cellular material.

The wall material can form a film that is cohesive with the core active ingredient. A wide variety of coating materials are available for encapsulation, e.g., traditional coating materials like inert polymers and pH sensitive ones as carboxylate and amino derivatives, which swell or dissolve according to the degree of cross-linking; some innovative coating polymers have also been developed for applications particularly among the bioadhesives and mucoadhesives. In some cases, the coating material can be hydrophilic polymers, hydrophobic polymers or a combination of both. In some cases, a microcapsule shell can comprise an amphipathic molecule. In some cases, the coating material can be gelatin, polyvinyl alcohol, ethyl cellulose, cellulose acetate phthalate and styrene maleic anhydride. In some instances, the coating material may not react with the pharmaceutical ingredient. In some cases, a microcapsule shell can comprise a trehalose. In some cases, a trehalose can comprise a trehalose dihydrate, a trehalose 6-decanoate, a trehalose 6-octanoate, a trehalose 6-tetradecanoate, a trehalose 6-dodecanoate, a trehalose 6-hexadecanoate, a D-(+)-trehalose dihydrate, an α,α-trehalose, an α,β-trehalose, a β,β-trehalose, a salt of a trehalose, or a mixture of any of these. In some cases, a microcapsule shell can comprise Hydroxypropyl methylcellulose (“HPMC”), Hydroxypropyl methylcellulose Acetate Succinate (“HPMCAS”), a cyclodextrin, maltodextrin, povidone, copovidone and others In some instances, a microcapsule shell can comprise HPMCAS-LG, HPMCAS-MG, HPMCAS-HG or HPMC-P or a combination thereof. In some instances, a microcapsule shell can comprise a different grade of HPMC or HPMCAS. For example, a microcapsule shell can comprise an F5, an F50, or a K4M grade of HPMC. In another example, a microcapsule shell can comprise a L, a M, or an H grade of HPMCAS. In some cases, a microcapsule shell can comprise a HPMCAS. In some cases, a microcapsule shell can comprise gelatin, cornstarch, polyvinylpyrrolidone (PVP), an oligosaccharide, a long chain sugar or any combination thereof. In some cases, a microcapsule shell can comprise a fatty acid, a liposome, an amino acid, a natural oil and a sugar, trehalose, dextran, a natural oil, a synthetic oil or a combination thereof. In some instances, an amino acid can comprise glutamic acid, aspartic acid, lysine, tryptophan, tyrosine, methionine or a combination thereof. In some cases, a coating material may not comprise diketopiperazine, leucine, trehalose, distearoylphosphatidylcholine (DSPC) or a combination thereof. In some cases, a fatty acid can comprise a polyunsaturated fatty acid, an essential fatty acid, a conjugated fatty acid, a short chain fatty acid, a medium chain fatty acid, a long chain fatty acid, a very long chain fatty acid, a saturated fatty acid, an unsaturated fatty acid, a monounsaturated fat, or any combination thereof. In some cases, a fatty acid can comprise an omega-3, an omega-5 fatty acid, an omega-6, an omega-7 fatty acid, an omega-9 fatty acid, an omega-10 fatty acid, an omega-11 fatty acid, an omega-12 fatty acid, or a combination thereof. In some cases, cellular material can be mixed or blended with an amphipathic molecule, such as a liposome, a triglyceride, an alpha linoleic acid (ALA), a phospholipid, an amino acid, an ethyl cellulose, a carboxymethylcellulose (CMC), a natural oil, a natural sugar, a trehalose, a dextran, a polysaccharide, a cellulose, a synthetic oil, a synthetic sugar, or any combination thereof. In some cases, a natural oil can comprise soybean oil, a vegetable oil, a food oil, evening primrose oil, borage oil, blackcurrant seed oil, flax or linseed oil, rapeseed or canola oil, corn oil, almond oil, avocado oil, brazil nut oil, canola oil, cashew oil, chia seed oil, cocoa butter oil, coconut oil, corn oil, cottonseed oil, flaxseed/linseed oil, grape seed oil, hemp seed oil, Vigna mungo oil, mustard oil, olive oil, palm oil, peanut oil, pecan oil, perilla oil, rice bran oil, safflower oil, sesame oil, soybean oil, walnut oil, sunflower oil, cottonseed oil, palm oil, or a combination thereof.

In some cases, a microcapsule shell for a cellular material can comprise a methyl cellulose, an ethyl cellulose, a hydroxy ethyl cellulose, a sodium carboxy methyl cellulose (CMC), a poly vinyl alcohol, a hypromellose phthalate (HPMCP), a povidone (e.g., poly vinyl pyrrolidone), a copovidone, a crospovidone, an enteric coatings of a methylacrylate copolymer, an amorphous silica gel, a synthetic phospholipid, a liposome, or a mixture thereof.

In some embodiments, a polymer coating an or an enteric coating comprises a barrier, such as a polymer barrier, that can be applied to a composition (for example a microencapsulated particle and/or a cellular material) to prevent dissolution or disintegration in the stomach. In some cases, this can enable the active ingredient to bypass the stomach to the small intestines before the active ingredient is released. In some cases, a wall material such as an additional coating on a previously microencapsulated particle can comprise an enteric coating. In some cases, an enteric coating can comprise a polymer. In some cases, an enteric coating can comprise methyl methacrylate (MMA). In some cases, an enteric coating can comprise a plant fiber, a shellac, a wax, a fatty acid, a plastic, or a combination thereof. In some cases, an enteric coating can comprise a methyl acrylate-methacrylic acid copolymer, a cellulose acetate phthalate (CAP), a cellulose acetate succinate, hydroxypropyl methyl cellulose phthalate, a HPMC-AS), a polyvinyl acetate phthalate (PVAP), a methyl methacrylate-methacrylic acid copolymer, a shellac, a cellulose acetate trimellitate, a sodium alginate, a zein, an enteric coating solution (an ethylcellulose, a medium chain triglycerides, an oleic acid, a sodium alginate, a stearic acid), or a combination thereof.

In some cases, a microcapsule shell can increase or decrease active ingredient release kinetics. In some cases, a microcapsule shell can increase or decrease bioavailability. In some cases, microencapsulation of a cellular material or a salt thereof can produce about: 5% to about 70%, 5% to about 10%, 5% to about 20%, 10% to about 30%, 15% to about 40%, 25% to about 40%, 10% to about 60%, or 20% to about 50% more bioavailability of the cellular material or the salt thereof as compared to the cellular material or the salt thereof that is not encapsulated when inhaled as a dry powdered composition by a subject. The wall material can be biodegradable and biocompatible with the pharmaceutical ingredient. In some cases, a microcapsule can be produced by dissolving or mixing the pharmaceutical ingredient in a solvent or aqueous solution containing the shell material to produce a liquid suspension. For example, trehalose can be dissolved with water and a cellular material (e.g., the core) can be added the liquid suspension. In some instances, the pharmaceutical compound may not dissolve in the liquid suspension. In some instances, the pharmaceutical compound may dissolve in the liquid suspension. The liquid suspension can be dried with a spray drying technique described herein or by another method.

For example, to protect a human cell it can be microencapsulated with a cellular material such as HPMC, HPMCAS, or a mixture thereof. In some cases, the cellular material once blended, mixed or both with an amphipathic molecule can create a dispersion that can be fed into the spray dry system. In one example, the characteristics of HPMCAS after spray drying can result in the hard outer shell (hydrophobic or polar) part and interior (hydrophilic non-polar).

In some cases, the average wall thickness of a microencapsulated particle can be of less than a value, of greater than a value, of at least a value, of a value, or ranging from any two values, wherein the value is selected from: about 500 nm, about 550 nm, about 600 nm, about about 650 nm, about 700 nm, about 750 nm, about 800 nm, about 850 nm, about 900 nm, about 950 nm, about 1 μm, about 2 μm, about 3 μm, about 4 μm, about 5 μm, about 6 μm, about 7 μm, about 8 μm, about 9 μm, about 10 μm, about 11 μm, about 12 μm, about 13 μm, about 14 μm, about 15 μm, about 16 μm, about 17 μm, about 18 μm, about 19 μm, about 20 μm, about 21 μm, about 22 μm, about 23 μm, about 24 μm, about 25 μm, about 26 μm, about 27 μm, about 28 μm, about 29 μm, or about 30 μm. In some cases, the wall thickness of a microencapsulated particle can range from about: 1 μm to about 10 μm, 1 μm to about 5 μm, 2 μm to about 7 μm, 3 μm to about 8 μm, 5 μm to about 10 μm, 5 μm to about 15 μm, or 1 μm to about 30 μm. In some instances, the wall thickness of a microencapsulated particle can increase by increasing the ratio of the wall material to the core material prior to spray drying. In some cases, the ratio of wall material to core material (weight/weight) can be about: 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 60:1, 70:1, 80:1, 90:1, or 100:1. In some cases, the ratio of the wall material to core material (weight/weight) can be about 10:1. In some cases, a microencapsulation process can use an amphipathic molecule to coat the cellular material. In some cases, ratios of about: 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, or 20:1 of an amphipathic material to a cellular material can be used for microencapsulation. In some cases, a higher ratio of an amphipathic material to a cellular material can result in an improved coating and more protection for the cellular material. In some cases, ratios of about: 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, or 20:1 of a cellular material to an amphipathic material can be used for microencapsulation.

In some embodiments, in a plurality of microencapsulated particles about: 1%, 5%, 10%, 15%, 20%, 25%, 30%, 35%, 40%, 45%, 50%, 55%, 60%, 65%, 70%, 75%. 80%, 90%, 95%, or 99% of the microencapsulated particles can comprise a core substantially encapsulated by a wall material. In some cases, in a plurality of microencapsulated particles all of the microencapsulated particles can comprise a core substantially encapsulated by a wall material. In some cases, in a plurality of microencapsulated particles about: 1% to about 50%, 1% to about 20%, 1% to about 10%, 5% to about 25%, 10% to about 40%, 10% to about 60%, 20% to about 70%, 20% to about 50%, 30% to about 80%, 40% to about 90%, 50% to about 75%, 60% to about 80%, 70% to about 90%, 75% to about 95%, 80% to about 90%, 80% to about 99%, 85% to about 100%, or 90% to about 100% of the microencapsulated particles can comprise a core substantially encapsulated by a wall material. In some cases, in a plurality of microencapsulated particles not all of the core material can be encapsulated by the wall material.

In some embodiments, microencapsulated particles have a mean, a median, or a mode particle diameter of less than a value, of greater than a value, of at least a value, of a value, or ranging from any two values, wherein the value is selected from: about 500 nm, about 1 μm, about 2 μm, about 3 μm, about 4 μm, about 5 μm, about 6 μm, about 7 μm, about 8 μm, about 9 μm, about 10 μm, about 11 μm, about 12 μm, about 13 μm, about 14 μm, about 15 μm, about 16 μm, about 17 μm, about 18 μm, about 19 μm, about 20 μm, about 21 μm, about 22 μm, about 23 μm, about 24 μm, about 25 μm, about 26 μm, about 27 μm, about 28 μm, about 29 μm, about 30 μm, about 31 μm, about 32 μm, about 33 μm, about 34 μm, about 35 μm, about 36 μm, about 37 μm, about 38 μm, about 39 μm, about 40 μm, about 41 μm, about 42 μm, about 43 μm, about 44 μm, about 45 μm, about 46 μm, about 47 μm, about 48 μm, about 49 μm, about 50 μm, about 51 μm, about 52 μm, about 53 μm, about 54 μm, about 55 μm, about 56 μm, about 57 μm, about 58 μm, about 59 μm, about 60 μm, about 61 μm, about 62 μm, about 63 μm, about 64 μm, about 65 μm, about 66 μm, about 67 μm, about 68 μm, about 69 μm, about 70 μm, about 71 μm, about 72 μm, about 73 μm, about 74 μm, about 75 μm, about 76 μm, about 77 μm, about 78 μm, about 79 μm, about 80 μm, about 81 μm, about 82 μm, about 83 μm, about 84 μm, about 85 μm, about 86 μm, about 87 μm, about 88 μm, about 89 μm, about 90 μm, about 91 μm, about 92 μm, about 93 μm, about 94 μm, about 95 μm, about 96 μm, about 97 μm, about 98 μm, about 99 μm, about 100 μm, about 101 μm, about 102 μm, about 103 μm, about 104 μm, about 105 μm, about 106 μm, about 107 μm, about 108 μm, about 109 μm, about 110 μm, about 111 μm, about 112 μm, about 113 μm, about 114 μm, about 115 μm, about 116 μm, about 117 μm, about 118 μm, about 119 μm, about 120 μm, about 121 μm, about 122 μm, about 123 μm, about 124 μm, about 125 μm, about 126 μm, about 127 μm, about 128 μm, about 129 μm, about 130 μm, about 131 μm, about 132 μm, about 133 μm, about 134 μm, about 135 μm, about 136 μm, about 137 μm, about 138 μm, about 139 μm, about 140 μm, about 141 μm, about 142 μm, about 143 μm, about 144 μm, about 145 μm, about 146 μm, about 147 μm, about 148 μm, about 149 μm, about 150 μm, about 151 μm, about 152 μm, about 153 μm, about 154 μm, about 155 μm, about 156 μm, about 157 μm, about 158 μm, about 159 μm, about 160 μm, about 161 μm, about 162 μm, about 163 μm, about 164 μm, about 165 μm, about 166 μm, about 167 μm, about 168 μm, about 169 μm, about 170 μm, about 171 μm, about 172 μm, about 173 μm, about 174 μm, about 175 μm, about 176 μm, about 177 μm, about 178 μm, about 179 μm, about 180 μm, about 181 μm, about 182 μm, about 183 μm, about 184 μm, about 185 μm, about 186 μm, about 187 μm, about 188 μm, about 189 μm, about 190 μm, about 191 μm, about 192 μm, about 193 μm, about 194 μm, about 195 μm, about 196 μm, about 197 μm, about 198 μm, about 199 μm, or about 200 μm. In some instances, microencapsulated particles have a mean, a median, or a mode particle diameter ranging from about: 500 nm to about 5 μm, 1 μm to about 10 μm, 1 μm to about 5 μm, 2 μm to about 7 μm, 3 μm to about 8 μm, 5 μm to about 10 μm, 5 μm to about 15 μm, 10 μm to about 50 μm, 20 μm to about 100 μm, 30 μm to about 70, 50 μm to about 150, 70 μm to about 140, 100 μm to about 180, or 120 μm to about 200 μm.

In some embodiments, the core material can be the material over which a coating has to be applied to serve the specific purpose. Core material may be in form of solids or droplets of liquids and dispersions. In some cases, the core material can comprise a cellular material. In some cases, the core material can comprise an individual cellular material of 2 or more cellular materials. The composition of core material can vary and thus furnish definite flexibility and allow effectual design and development of the desired microcapsule properties.

A substance may be microencapsulated for a number of reasons. Examples may include protection of reactive material from their environment, safe and convenient handling of the materials which can be otherwise toxic or noxious, taste masking, means for controlled or modified release properties, means of handling liquids as solids, preparation of free flow powders, and in modification of physical properties of the drug. For example, encapsulation can improve solubility and dissolution and therefore increase bioavailability of an active ingredient such as a cellular material. In some instances, microencapsulation can be used to increase the stability, limit evaporation of the cellular core, and/or improve the handling properties and isolate for storage. In some instances, microencapsulation can facilitate higher bioavailability when reconstituted or administered. Microencapsulation can be used to increase the stability, improve the handling properties of compounds, facilitate higher bioavailability when reconstituted or administered. In some cases, spray drying can improve therapeutic efficacy, safety, tolerability, patient compliance or any combination thereof. In some cases, spray drying can be fast, continuous, cost effective and/or can create a lower dosage cost. In some cases, spray drying can achieve a higher concentration of an active ingredient in a packaging (per weight amount) as compared to a comparable non-spray dried material in a packaging.

In some instances, the core diameter of a microencapsulated particle can be of less than a value, of greater than a value, of at least a value, of a value, or ranging from any two values, wherein the value is selected from: about 100 nm (nanometer), about 150 nm, about 200 nm, about 250 nm, about 300 nm, about 350 nm, about 400 nm, about 450 nm, about 500 nm, about 550 nm, about 600 nm, about 650 nm, about 700 nm, about 750 nm, about 800 nm, about 850 nm, about 900 nm, about 950 nm, about 1 μm, about 2 μm, about 3 μm, about 4 μm, about 5 μm, about 6 μm, about 7 μm, about 8 μm, about 9 μm, about 10 μm, about 11 μm, about 12 μm, about 13 μm, about 14 μm, about 15 μm, about 16 μm, about 17 μm, about 18 μm, about 19 μm, about 20 μm, about 21 μm, about 22 μm, about 23 μm, about 24 μm, about 25 μm, about 26 μm, about 27 μm, about 28 μm, about 29 μm, about 30 μm, about 31 μm, about 32 μm, about 33 μm, about 34 μm, about 35 μm, about 36 μm, about 37 μm, about 38 μm, about 39 μm, about 40 μm, about 41 μm, about 42 μm, about 43 μm, about 44 μm, about 45 μm, about 46 μm, about 47 μm, about 48 μm, about 49 μm, about 50 μm, about 51 μm, about 52 μm, about 53 μm, about 54 μm, about 55 μm, about 56 μm, about 57 μm, about 58 μm, about 59 μm, about 60 μm, about 61 μm, about 62 μm, about 63 μm, about 64 μm, about 65 μm, about 66 μm, about 67 μm, about 68 μm, about 69 μm, about 70 μm, about 71 μm, about 72 μm, about 73 μm, about 74 μm, about 75 μm, about 76 μm, about 77 μm, about 78 μm, about 79 μm, about 80 μm, about 81 μm, about 82 μm, about 83 μm, about 84 μm, about 85 μm, about 86 μm, about 87 μm, about 88 μm, about 89 μm, about 90 μm, about 91 μm, about 92 μm, about 93 μm, about 94 μm, about 95 μm, about 96 μm, about 97 μm, about 98 μm, about 99 μm, about 100 μm, about 101 μm, about 102 μm, about 103 μm, about 104 μm, about 105 μm, about 106 μm, about 107 μm, about 108 μm, about 109 μm, about 110 μm, about 111 μm, about 112 μm, about 113 μm, about 114 μm, about 115 μm, about 116 μm, about 117 μm, about 118 μm, about 119 μm, about 120 μm, about 121 μm, about 122 μm, about 123 μm, about 124 μm, about 125 μm, about 126 μm, about 127 μm, about 128 μm, about 129 μm, about 130 μm, about 131 μm, about 132 μm, about 133 μm, about 134 μm, about 135 μm, about 136 μm, about 137 μm, about 138 μm, about 139 μm, about 140 μm, about 141 μm, about 142 μm, about 143 μm, about 144 μm, about 145 μm, about 146 μm, about 147 μm, about 148 μm, about 149 μm, about 150 μm, about 151 μm, about 152 μm, about 153 μm, about 154 μm, about 155 μm, about 156 μm, about 157 μm, about 158 μm, about 159 μm, about 160 μm, about 161 μm, about 162 μm, about 163 μm, about 164 μm, about 165 μm, about 166 μm, about 167 μm, about 168 μm, about 169 μm, about 170 μm, about 171 μm, about 172 μm, about 173 μm, about 174 μm, about 175 μm, about 176 μm, about 177 μm, about 178 μm, about 179 μm, about 180 μm, about 181 μm, about 182 μm, about 183 μm, about 184 μm, about 185 μm, about 186 μm, about 187 μm, about 188 μm, about 189 μm, or about 190 μm. In some cases, the core diameter of a microencapsulated particle can range from about: 100 nm to about 250 nm, 100 nm to about 500 nm, 100 nm to about 1 μm, 500 nm to about 5 μm, 1 μm to about 10 μm, 1 μm to about 5 μm, 2 μm to about 7 μm, 3 μm to about 8 μm, 5 μm to about 10 μm, 5 μm to about 15 μm, 10 μm to about 50 μm, 20 μm to about 100 μm, 30 μm to about 70, 50 μm to about 150, 70 μm to about 140, 100 μm to about 180, or 120 μm to about 190 μm. In some instances, the core can comprise about: 1%, 5%, 10%, 15%, 20%, 25%, 30%, 35%, 40%, 45%, 50%, 55%, 60%, 65%, 70%, 75%. 80%, 90%, 95% or 99% of the total microcapsule content (e.g., total weight of the core and wall material). In some instances, the core can comprise about: 1% to about 50%, 1% to about 20%, 1% to about 10%, 5% to about 25%, 10% to about 40%, 10% to about 60%, 20% to about 70%, 20% to about 50%, 30% to about 80%, 40% to about 90%, 50% to about 75%, or 1% to about 99% of the total microcapsule content.

In some cases, microencapsulation of a cellular material or a salt thereof by HPMCAS can provide faster absorption in the lungs. For example, a lipid may not be water soluble and microencapsulation with HPMCAS can provide increased absorption into the blood stream from the lungs. In some instances, microencapsulation can increase the solubility of an active ingredient. In some cases, a microencapsulated cellular material or a salt thereof may be absorbed about: 10% to about 70%, 5% to about 10%, 5% to about 20%, 10% to about 30%, 15% to about 40%, 25% to about 40%, 35% to about 50%, 10% to about 60%, 40% to about 90%, or 20% to about 50% faster than a cellular material that is not microencapsulated. In some cases, a microencapsulated cellular material or a salt thereof may be absorbed after inhalation into the blood stream in about: 5 seconds to about 30 seconds, 5 seconds to about 20 seconds, 10 seconds to about 20 seconds, 10 seconds to about 30 seconds, 10 seconds to about 60 seconds, 20 seconds to about 40 seconds, 30 second to about 60 seconds, 30 seconds to about 2 minutes, or 1 minute to about 2 minutes.

In some embodiments, a method of microencapsulation can comprise at least partially dissolving or suspending the coating material (e.g., HPMC or HPMCAS) in a solvent and/or water. In some cases, a cellular material can be micronized with a micronizer to generate small liquid droplets. In some cases, a cellular material, such as a cell disclosed herein, may not be micronized with a micronizer (or any system that could damage a cell). In some cases, a microfluidic system can be used to generate small liquid droplets. In some cases, the liquid droplets may be an oil. In some cases, the liquid droplets may be aqueous The liquid droplets can be added to the solution of the coating material and the solvent and/or water to create a suspension of the liquid droplets and the coating material. In some instances, the liquid droplets may not dissolve in the suspension and may remain in suspension. The suspension can be mixed to an at least partially uniform mixture and spray dried. The coating can at least partially encapsulate the liquid droplets containing the cellular material or salt thereof. In some cases, the encapsulation of a cellular material can be a spherical, round, oval, or any shape structure.

### Methods of Making the Powdery Pharmaceutical Composition to Select Sizes

In some embodiments, a method of making the powdery pharmaceutical composition can comprise mixing particles of a pharmaceutically acceptable excipient and particles comprising an active ingredient or a pharmaceutically acceptable salt thereof at least partially encapsulated in a coating material and wherein the particles at least partially encapsulated in the coating material are spray dried. In some instances, a method of making the powdery pharmaceutical composition can comprise mixing particles in a mixer.

In some embodiments, the method of making the powdery pharmaceutical composition can comprise mixing the particles described herein. In some instances, at least a portion of the particles of the pharmaceutically acceptable excipient can have a particle diameter ranging from about 50 μm to about 200 μm, as measured by a particle size analyzer using laser diffraction; and particles comprising an active ingredient or a pharmaceutically acceptable salt thereof at least partially encapsulated in a coating material can have a particle diameter ranging from about 1 μm to about 100 μm, 10 μm to about 50 μm, or from about 1 μm to about 200 μm, as measured by a particle size analyzer using laser diffraction.

In some embodiments, at least a portion of the particles of the pharmaceutically acceptable excipient can have a particle diameter ranging from about: 30 μm to about 60 μm, 50 μm, to about 200 μm, 60 μm to about 80 μm, 70 μm to about 100 μm, 90 μm to about 130 μm, 110 μm to about 150 μm, 130 μm to about 180 μm, 150 μm to about 200 μm, 190 μm to about 250 μm, or 200 μm to about 400 μm. In some cases, particles of the pharmaceutically acceptable excipient can have a particle diameter of less than a value, of greater than a value, of at least a value, of a value, or ranging from any two values, wherein the value is selected from: about 30 μm, about 40 μm, about 45 μm, about 50 μm, about 55 μm, about 60 μm, about 65 μm, about 70 μm, about 75 μm, about 80 μm, about 85 μm, about 90 μm, about 95 μm, about 100 μm, about 105 μm, about 110 μm, about 120 μm, about 130 μm, about 140 μm, about 150 μm, about 160 μm, about 170 μm, about 180 μm, about 190 μm, about 200 μm, about 210 μm, about 220 μm, about 230 μm, about 240 μm, about 250 μm, about 260 μm, about 270 μm, about 280 μm, about 290 μm, about 300 μm, about 310 μm, about 320 μm, about 330 μm, about 340 μm, about 350 μm, about 360 μm, about 370 μm, about 380 μm, about 390 μm, or about 400 μm. In some cases, the particles of a pharmaceutically acceptable excipient can range from about 50 μm to about 100 μm, which may be preferred when inhaled or administered intranasally for deposit on the oropharynx. In some instances, particle size can comprise the diameter, the radius, or length of a particle. In some instances, particle size can be a measure of the mean, the median or the mode of a plurality of particles.

In some embodiments, particles comprising an active ingredient or a pharmaceutically acceptable salt thereof at least partially encapsulated in a coating material can have particle diameters of less than a value, of greater than a value, of at least a value, of a value, or ranging from any two values, wherein the value is selected from: about 500 nm (nanometer), about 1 μm, about 2 μm, about 3 μm, about 4 μm, about 5 μm, about 6 μm, about 7 μm, about 8 μm, about 9 μm, about 10 μm, about 11 μm, about 12 μm, about 13 μm, about 14 μm, about 15 μm, about 16 μm, about 17 μm, about 18 μm, about 19 μm, about 20 μm, about 21 μm, about 22 μm, about 23 μm, about 24 μm, about 25 μm, about 26 μm, about 27 μm, about 28 μm, about 29 μm, about 30 μm, about 31 μm, about 32 μm, about 33 μm, about 34 μm, about 35 μm, about 36 μm, about 37 μm, about 38 μm, about 39 μm, about 40 μm, about 41 μm, about 42 μm, about 43 μm, about 44 μm, about 45 μm, about 46 μm, about 47 μm, about 48 μm, about 49 μm, about 50 μm, about 51 μm, about 52 μm, about 53 μm, about 54 μm, about 55 μm, about 56 μm, about 57 μm, about 58 μm, about 59 μm, about 60 μm, about 61 μm, about 62 μm, about 63 μm, about 64 μm, about 65 μm, about 66 μm, about 67 μm, about 68 μm, about 69 μm, about 70 μm, about 71 μm, about 72 μm, about 73 μm, about 74 μm, about 75 μm, about 76 μm, about 77 μm, about 78 μm, about 79 μm, about 80 μm, about 81 μm, about 82 μm, about 83 μm, about 84 μm, about 85 μm, about 86 μm, about 87 μm, about 88 μm, about 89 μm, about 90 μm, about 91 μm, about 92 μm, about 93 μm, about 94 μm, about 95 μm, about 96 μm, about 97 μm, about 98 μm, about 99 μm, about 100 μm, about 101 μm, about 102 μm, about 103 μm, about 104 μm, about 105 μm, about 106 μm, about 107 μm, about 108 μm, about 109 μm, about 110 μm, about 111 μm, about 112 μm, about 113 μm, about 114 μm, about 115 μm, about 116 μm, about 117 μm, about 118 μm, about 119 μm, about 120 μm, about 121 μm, about 122 μm, about 123 μm, about 124 μm, about 125 μm, about 126 μm, about 127 μm, about 128 μm, about 129 μm, about 130 μm, about 131 μm, about 132 μm, about 133 μm, about 134 μm, about 135 μm, about 136 μm, about 137 μm, about 138 μm, about 139 μm, about 140 μm, about 141 μm, about 142 μm, about 143 μm, about 144 μm, about 145 μm, about 146 μm, about 147 μm, about 148 μm, about 149 μm, about 150 μm, about 151 μm, about 152 μm, about 153 μm, about 154 μm, about 155 μm, about 156 μm, about 157 μm, about 158 μm, about 159 μm, about 160 μm, about 161 μm, about 162 μm, about 163 μm, about 164 μm, about 165 μm, about 166 μm, about 167 μm, about 168 μm, about 169 μm, about 170 μm, about 171 μm, about 172 μm, about 173 μm, about 174 μm, about 175 μm, about 176 μm, about 177 μm, about 178 μm, about 179 μm, about 180 μm, about 181 μm, about 182 μm, about 183 μm, about 184 μm, about 185 μm, about 186 μm, about 187 μm, about 188 μm, about 189 μm, about 190 μm, about 191 μm, about 192 μm, about 193 μm, about 194 μm, about 195 μm, about 196 μm, about 197 μm, about 198 μm, about 199 μm, or about 200 μm. In some cases, particles comprising an active ingredient or a pharmaceutically acceptable salt thereof at least partially encapsulated in a coating material can have a particle diameter ranging from about: 500 nm to about 5 μm, 1 μm to about 10 μm, 1 μm to about 5 μm, 2 μm to about 7 μm, 3 μm to about 8 μm, 5 μm to about 10 μm, 5 μm to about 15 μm, 10 μm to about 50 μm, 20 μm to about 100 μm, 30 μm to about 70, 50 μm to about 150, 70 μm to about 140, 100 μm to about 180, or 120 μm to about 200 μm. In some cases, particles comprising an active ingredient or a pharmaceutically acceptable salt thereof at least partially encapsulated in a coating material can have a particle diameter can have a particle diameter of about 1 μm to about 5 μm, which can be preferred when inhaled or administered intranasally for absorption into lung alveoli.

In some embodiments, particles or compositions described herein can have a tap density of more than about: 0.1 grams/centimeter³ (g/cm³), 0.2 g/cm³, 0.3 g/cm³, 0.4 g/cm³, 0.5 g/cm³, 0.6 g/cm³, 0.7 g/cm³, 0.8 g/cm³, 0.9 g/cm³, 1.0 g/cm³, 1.1 g/cm³, or 1.2 g/cm³. In some instances, particles described herein can have a tap density of less than about: 0.1 g/cm³, 0.2 g/cm³, 0.3 g/cm³, 0.4 g/cm³, 0.5 g/cm³, 0.6 g/cm³, 0.7 g/cm³, 0.8 g/cm³, 0.9 g/cm³, 1.0 g/cm³, 1.1 g/cm³, or 1.2 g/cm³. In some cases, particles or compositions described herein can have a tap density of more than about 0.6 g/cm³, 0.7 g/cm³. In some cases, tap density can be a measure of the envelope mass density characterizing a particle. The envelope mass density of a particle of a statistically isotropic shape can be defined as the mass of the particle divided by the minimum sphere envelope volume within which it can be enclosed. Features which can contribute to low tap density include irregular surface texture, porous structure or a combination thereof. Tap density can be measured by using instruments known to those skilled in the art such as the Dual Platform Microprocessor Controlled Tap Density Tester (Vankel, N.C.) or a GeoPyc™ instrument (Micrometrics Instrument Corp., Norcross, Ga.).

In some embodiments, particles comprising an active ingredient or a pharmaceutically acceptable salt thereof can be mixed in sizes. In some cases, the mixed sizes can change the release time of the drug. For example, encapsulated particles with small sizes (e.g. about 1 μm to about 5 μm) can be readily absorbed into the blood stream while encapsulated particles larger than about 10 μm can take longer to be absorbed into the blood stream. In some cases, particles with diameters of about 1 μm to about 10 μm can be inhaled into the lung while larger particles may be deposited onto the oropharynx. In some cases, particles with diameters of about 1 μm to about 5 μm can absorb faster than particles with diameters of about 7 μm to about 10 μm. In some instances, the particles with sizes of about 7 μm to about 10 μm can be mixed with particles with sizes of about 1 μm to about 5 μm. In some instances, the weight to weight ratio of the particles with diameters of about 7 μm to about 10 μm to the particles with sizes of about 1 μm to about 5 μm can range from about 1:1 to about 1:2, about 1:1 to about 1:3, about 1:1 to about 1:4, about 1:1 to about 1:5, about 1:1 to about 1:8, about 1:1 to about 1:10, about 1:2 to about 1:3, about 1:2 to about 1:4, about 1:2 to about 1:5, about 1:2 to about 1:8, about 1:2 to about 1:10, about 1:3 to about 1:4, about 1:3 to about 1:5, about 1:3 to about 1:8, about 1:3 to about 1:10, about 1:4 to about 1:5, about 1:4 to about 1:8, about 1:4 to about 1:10, about 1:5 to about 1:8, about 1:5 to about 1:10, or 1:8 to about 1:10. In some instances, the weight to weight ratio of the particles with diameters of about 1 μm to about 5 μm to the particles with sizes of about 7 μm to about 10 μm can range from about 1:1 to about 1:2, about 1:1 to about 1:3, about 1:1 to about 1:4, about 1:1 to about 1:5, about 1:1 to about 1:8, about 1:1 to about 1:10, about 1:2 to about 1:3, about 1:2 to about 1:4, about 1:2 to about 1:5, about 1:2 to about 1:8, about 1:2 to about 1:10, about 1:3 to about 1:4, about 1:3 to about 1:5, about 1:3 to about 1:8, about 1:3 to about 1:10, about 1:4 to about 1:5, about 1:4 to about 1:8, about 1:4 to about 1:10, about 1:5 to about 1:8, about 1:5 to about 1:10, or 1:8 to about 1:10. In some instances, the particles with larger sizes (about 10 μm to about 20 μm) can be mixed with particles with smaller sizes (about 1 μm to about 10 μm). In some instances, the weight to weight ratio of the particles with larger sizes (about 10 μm to about 20 μm) to the particles with smaller sizes (about 1 μm to about 10 μm) can be range from about 1:1 to about 1:2, about 1:1 to about 1:3, about 1:1 to about 1:4, about 1:1 to about 1:5, about 1:1 to about 1:8, about 1:1 to about 1:10, about 1:2 to about 1:3, about 1:2 to about 1:4, about 1:2 to about 1:5, about 1:2 to about 1:8, about 1:2 to about 1:10, about 1:3 to about 1:4, about 1:3 to about 1:5, about 1:3 to about 1:8, about 1:3 to about 1:10, about 1:4 to about 1:5, about 1:4 to about 1:8, about 1:4 to about 1:10, about 1:5 to about 1:8, about 1:5 to about 1:10, or 1:8 to about 1:10. In some instances, the weight to weight ratio of the particles with smaller sizes (about 1 μm to about 10 μm) to the particles with larger sizes (about 10 μm to about 20 μm) can be range from about 1:1 to about 1:2, about 1:1 to about 1:3, about 1:1 to about 1:4, about 1:1 to about 1:5, about 1:1 to about 1:8, about 1:1 to about 1:10, about 1:2 to about 1:3, about 1:2 to about 1:4, about 1:2 to about 1:5, about 1:2 to about 1:8, about 1:2 to about 1:10, about 1:3 to about 1:4, about 1:3 to about 1:5, about 1:3 to about 1:8, about 1:3 to about 1:10, about 1:4 to about 1:5, about 1:4 to about 1:8, about 1:4 to about 1:10, about 1:5 to about 1:8, about 1:5 to about 1:10, or 1:8 to about 1:10. In some instances, the particles with larger sizes (about 100 μm to about 200 μm) can be mixed with particles with smaller sizes (about 30 μm to about 80 μm). In some instances, the weight to weight ratio of the particles with larger sizes (about 100 μm to about 200 μm) to the particles with smaller sizes (about 30 μm to about 80 μm) can be range from about 1:1 to about 1:2, about 1:1 to about 1:3, about 1:1 to about 1:4, about 1:1 to about 1:5, about 1:1 to about 1:8, about 1:1 to about 1:10, about 1:2 to about 1:3, about 1:2 to about 1:4, about 1:2 to about 1:5, about 1:2 to about 1:8, about 1:2 to about 1:10, about 1:3 to about 1:4, about 1:3 to about 1:5, about 1:3 to about 1:8, about 1:3 to about 1:10, about 1:4 to about 1:5, about 1:4 to about 1:8, about 1:4 to about 1:10, about 1:5 to about 1:8, about 1:5 to about 1:10, or about 1:8 to about 1:10. In some instances, the weight to weight ratio of the particles with smaller sizes (about 30 μm to about 80 μm) to the particles with larger sizes (about 100 μm to about 200 μm) can be range from about 1:1 to about 1:2, about 1:1 to about 1:3, about 1:1 to about 1:4, about 1:1 to about 1:5, about 1:1 to about 1:8, about 1:1 to about 1:10, about 1:2 to about 1:3, about 1:2 to about 1:4, about 1:2 to about 1:5, about 1:2 to about 1:8, about 1:2 to about 1:10, about 1:3 to about 1:4, about 1:3 to about 1:5, about 1:3 to about 1:8, about 1:3 to about 1:10, about 1:4 to about 1:5, about 1:4 to about 1:8, about 1:4 to about 1:10, about 1:5 to about 1:8, about 1:5 to about 1:10, or about 1:8 to about 1:10.

In some embodiments, active ingredient particles can be produced by spray drying. In some cases, encapsulated active ingredient particles can be produce by spray drying. In some instances, active ingredient particles can be produced by another method. In some instances, active ingredient particles can be produced by air-jet micronization, spiral milling, controlled precipitation, high-pressure homogenization, or cryo-milling

In some embodiments, particles that are not of the pharmaceutically acceptable excipient, can have particle diameters ranging from about 1 μm to about 20 μm. In some instances, particle diameters can be measured by a particle analyzer using laser diffraction (LD), static light scattering, dynamic light scattering (DLS), or nanoparticle tracking analysis (NTA).

### Packaging of the Powdery Pharmaceutical Compositions

In some embodiments, the pharmaceutical composition can be contained within a capsule, a tablet, a gel, a gummy, a spray, an ointment, a paste, a jelly, an oil, a butter, a tincture, a lotion, a cream, a balm, a food, a drink, a liquid, a syrup, or any combination thereof. In some cases, a capsule can be a capsule-in-capsule.

In some embodiments, microencapsulated particles as a food or drink additive can have a mean, a median, or a mode particle diameter of less than a value, of greater than a value, of at least a value, of a value, or ranging from any two values, wherein the value is selected from: about 45 μm, about 46 μm, about 47 μm, about 48 μm, about 49 μm, about 50 μm, about 51 μm, about 52 μm, about 53 μm, about 54 μm, about 55 μm, about 56 μm, about 57 μm, about 58 μm, about 59 μm, about 60 μm, about 61 μm, about 62 μm, about 63 μm, about 64 μm, about 65 μm, about 66 μm, about 67 μm, about 68 μm, about 69 μm, about 70 μm, about 71 μm, about 72 μm, about 73 μm, about 74 μm, about 75 μm, about 76 μm, about 77 μm, about 78 μm, about 79 μm, about 80 μm, about 81 μm, about 82 μm, about 83 μm, about 84 μm, about 85 μm, about 86 μm, about 87 μm, about 88 μm, about 89 μm, about 90 μm, about 91 μm, about 92 μm, about 93 μm, about 94 μm, about 95 μm, about 96 μm, about 97 μm, about 98 μm, about 99 μm, about 100 μm, about 101 μm, about 102 μm, about 103 μm, about 104 μm, or about 105 μm.

In some embodiments, an encapsulated cellular material and an excipient can be admixed into a substantially homogenous mixture. In some cases, the blended powder (e.g., the cellular material and excipient) can be loaded into a hopper of an encapsulator machine which can feed the powder into the capsules. The encapsulator can automatically separate the capsule top (cap) and body (shell) and the powder can be slugged (liquids can be placed into the capsule with a piston type or rotary pump) and then transferred into the body of the capsule. The capsule halves can be closed together to form an enclosed capsule that contains the blended powder. In some cases, a capsule-in-capsule can comprise the cellular powder and excipient. In some instances, the active ingredient can be placed into a first hypromellose capsule. In some cases, another capsule containing an active ingredient can comprise the first hypromellose capsule to create a capsule-in-capsule composition. In some cases, during filling, the capsule atmosphere can be made inert with nitrogen to prevent oxidation and remove moisture from the blend so that inhalable powder can flow freely from the capsule using the dry powder inhaler. In some cases, the dry powder can be placed into a capsule (e.g., a hypromellose capsule), by a Bosch, ACG or IMA encapsulator machine.

In some embodiments a capsule band can be added to a capsule. In some cases, capsule banding can be the process of sealing the capsule so that it may be filled with liquids, powders or other types of ingredients. In some instances, there can be a seal joint between the capsule cap and body that can require an additional band to be applied to prevent leakage of the drug outside the capsule. In some instances, it can provide a tamper resistant band that can reduce oxidation and minimizes any odor. In some cases, the banding can be applied with a banding machine that applies a thin layer of HPMC (hydroxypropyl methylcellulose) as the capsules pass over two rollers which apply the capsule banding material. In this process, the banding material can be heated and temperature controlled to make a smooth, liquid-tight band that join the capsule top and body. This can provide a visual tamper resistant barrier on the capsule.

In some embodiments, the capsule may comprise a single-piece capsule, two-piece capsule, transparent capsule, non-transparent capsule, opaque capsule, slow-release capsule, extended-release capsule, standard-release capsule, rapid-release capsule, quick-release capsule, hard-shell capsule, soft gel capsule, gel capsule, hard gelatin capsule, soft gelatin capsule, animal-based capsule, vegetarian capsule, polysaccharide capsule, cellulose capsule, mucopolysaccharide capsule, tapioca capsule, hydroxypropylmethyl cellulose (HPMC) capsule, pullulan capsule, enteric capsule, uncoated capsule, coated capsule, capsule comprising titanium dioxide, fatty acids, waxes, shellac, plastics, pasticizers, glycerin, sorbitol, plant fibers, additives, preservatives, colorants, or any combination thereof. In some instances, the capsule may comprise a vegetarian capsule. In some embodiments, the capsule can comprise a capsule-in-capsule as described herein. For example, second capsule can comprise a first capsule.

In some embodiments, a capsule can further comprise a capsule coating. In some cases, a capsule coating can be added to a capsule to further improve stability (light protection, moisture and gas barrier), facilitate administration, or modify the composition release behavior from the dosage form. A capsule coating may be used to enable the immediate release of the composition, delay the release of the composition (such as in enteric coatings), or sustain the release of the composition over extended periods of time In some instances, a capsule coating can comprise a film coating, a gelatin coating, or both. In some instances, a capsule coating can comprise an enteric coating, a time release coating, a pH dependent coating, a delayed release coating, an extended-release coating, or a combination thereof. For example, an enteric coating can be added to a capsule to prevent it from dissolving until after it passes through the stomach. In some cases, the composition can release depending on the pH value within the gastrointestinal (GI) tract. The GI tract can have different pH values which can allow for pH dependent dosing in specific areas. For example, the pH of the stomach (acidic about 1.5-4.0 pH) is different from the pH of the small intestine (pH 4.0-7.0), and a pH coating can be used to dose areas of the GI tract with specific pH levels. In some instances, an enteric coating of a capsule can be a polymer barrier that can be applied to the capsules described herein to enable a controlled release. Bypassing the stomach can allow for more precise dosing and can enable the drug to achieve a higher bioavailability in the gastric tract. In some cases, these coatings can be modified to deliver medicine from the mouth, all the way to the colon. In some cases, the technology can be applied to the outer (e.g., the second capsule) and the inner (e.g., the first capsule) capsule in the capsule-in-capsule technology and utilize time-released, pH-controlled released, or a combination of both technologies to achieve the intended drug delivery. In some instances, an enteric coating can be applied to multiple capsules, for example to an inner capsule and to an outer capsule and to provide delayed release of both capsules. In some cases, a capsule coating can provide a color, mask a bitter taste, or both. In some cases, a capsule coating can comprise polymers, plasticizers, pigments, opacifiers, glidants, binders, anti-tacking agents, anti-foaming mechanisms, surfactants, fillers, and extenders.

In some embodiments, an enteric coating can comprise a polymer. In some cases, an enteric coating can comprise a methyl acrylate-methacrylic acid copolymer, a cellulose acetate phthalate, methyl methacrylate (MMA), a cellulose acetate succinate, a hydroxypropyl methyl cellulose phthalate, a hydroxypropyl methyl cellulose acetate succinate (hypromellose acetate succinate), a polyvinyl acetate phthalate, a methyl methacrylate-methacrylic acid copolymers, a shellac, a cellulose acetate trimellitate, a sodium alginate, a zein, an ethylcellulose, a medium chain triglycerides, an oleic acid, a stearic acid or any combination thereof.

In some embodiments, a capsule can be configured (for example with a capsule coating) to at least partially release an active ingredient in: the mouth, the esophagus, the small intestine, the duodenum, the jejunum, the ileum, the cecum, the colon, the ascending colon, the traverse colon, the descending colon, the sigmoid colon, the rectum, the anus, or any combination thereof.

In some embodiments, a capsule can comprise a pH buffering composition to alter the pH of the environment of the capsule when released. In some instances, a pH buffering composition can comprise an excipient. In some cases, an excipient can comprise a pH buffering composition. In some instances, a pH buffering composition can comprise sodium phosphate, citric acid, acetic acid, potassium phosphate, tromethamine, gluconic acid, lactic acid, tartaric acid, aspartic acid, glutamic acid, citric acid cycle intermediates (citrate, fumarate, a-ketoglutarate, malate and succinate) or any combination thereof.

In some embodiments, the capsule can have different sizes according to pharmaceutical composition requirements. In some instances, the capsule size can be size: Su07, 7, 10, 11, 12el, 12, 13, 000, 0E, 00, 0, 1, 2, 3, 4, or 5. In some instances, the capsule size can be 000. In some instances, the capsule size can be 00. In some instances, the capsule size can be 0. In some instances, the capsule size can be 1. In some instances, the capsule size can be 2. In some instances, the capsule size can be 3. In some instances, the capsule size can be 4. In some instances, a capsule size can be 5. In some instances, the capsule capacity varies from about 0.21 ml to about 1.37 ml. In some instances, the capsule capacity varies from about 0.13 ml (e.g. size 5) to about 28 ml. In some instances, a first capsule (e.g., the inner capsule) can be size: 000, 00, 0, 1, 2, 3, 4, or 5. In some instances, a second capsule (e.g., the outer capsule) can be size: 000, 00, 0, 1, 2, 3, or 4. In some cases, a capsule encapsulating another capsule will be different sizes. For example, an outer capsule can be larger (e.g., size 0 or size 00) than an inner capsule (e.g., size 1 or size 3) which it can encapsulate.

In some instances, the at least partially encapsulated cells can be stored at about 5° C., 10° C., 15° C., 20° C., 25° C., 30° C. or about 35° C. and at the end of at least 1 week, 2 weeks, 3 weeks, 4 weeks, 1 month, 2 months, 3 months, 4 months, 5 months, 6 months, 7 months, 8 months, 9 months, 10 months, 11 months, 12 months or more at least about: 0.1%, 1%, 10%, 50%, 60%, 70%, 80%, 90%, or about 100% of the live cells remain alive. In some cases, cells can be stored in a room with about 30-50% humidity. The storage can be in a sealed or an unsealed container. In some cases, a dry powder herein can be placed in an hermetically sealed container. In some cases, a dry powder herein can be stored in a sterile glass bottle. In some cases, an encapsulated cell or cellular component may not require refrigeration or cryogenic (e.g., freezer) storage. In some embodiments, after microencapsulation about: 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of microencapsulated cells are alive. In some cases, after microencapsulation all of the microencapsulated cells are alive. In some cases, after microencapsulation about: 0.01% to about 0.1%, 0.1% to about 1%, 1% to about 99%, 1% to about 10%, 1% to about 50%, 20% to about 80%, or 50% to about 100% of the microencapsulated cells are alive. In some instances, cell viability can be determined by enumerating colony-forming unit (CFU) or any other viability determining method, for example, trypan blue cell counting, live dead staining and/or fluorescence-activated single cell sorting (FACS).

In some embodiments, a composition can be contained within a capsule, wherein the capsule can be loaded with about 5% to about 99% or about 10% to about 75% (by volume) with the composition. In some cases, the capsule can be loaded with about: 5%, 10%, 15%, 20%, 25%, 30%, 31%, 32%, 33%, 34%, 35%, 36%, 37%, 38%, 39%, 40%, 45%, 50%, 55%, 60%, 65%, 70%, 75%, 80%, 85%, 90%, 95%, 99% (by volume) with a composition described herein. In some instances, the capsule can be loaded with about 5% to about 20%, about 20% to about 25%, about 20% to about 30%, about 20% to about 40%, about 20% to about 50%, about 20% to about 60%, about 20% to about 65%, about 20% to about 70%, about 20% to about 75%, 25% to about 30%, about 25% to about 40%, about 25% to about 50%, about 25% to about 60%, about 25% to about 65%, about 25% to about 70%, about 25% to about 75%, about 30% to about 40%, about 30% to about 50%, about 30% to about 60%, about 30% to about 65%, about 30% to about 70%, about 30% to about 75%, about 40% to about 50%, about 40% to about 60%, about 40% to about 65%, about 40% to about 70%, about 40% to about 75%, about 50% to about 60%, about 50% to about 65%, about 50% to about 70%, about 50% to about 75%, about 60% to about 65%, about 60% to about 70%, about 60% to about 75%, about 65% to about 70%, about 65% to about 75%, about 70% to about 75%, or about 75% to about 100% (by volume) with the composition. In some cases, a capsule can be loaded with another capsule to create a capsule-in-capsule composition.

In some embodiments, the content of the capsule comprises less than about: 10%, 9%, 8%, 7%, 6%, 5%, 4%, 3%, 2% or 1% water by weight. In some instances, the content of the capsule comprises less than about 95%, 90%, 85%, 80%, 75%, 70%, 50%, 40%, 30%, 25%, 20%, 10%, 5%, or 1% water by weight. In some instances, the content of the capsule comprises more than about 95%, 90%, 85%, 80%, 75%, 70%, 50%, 40%, 30%, 25%, 20%, 10%, 5%, or 1% water by weight.

In some embodiments, the total content of all gases in the capsule can be less than about: 10%, 9%, 8%, 7%, 6%, 5%, 4%, 3%, 2% or 1% water by weight. In some instances, the total content of all gases in the capsule can be less than about: 80%, 75%, 70%, 50%, 40%, 30%, 25%, 20%, 10%, 5%, or 1% water by weight.

In some embodiments, the capsule further comprises, in the volume not occupied by the powdery pharmaceutical composition, an inert gas. In some cases, the capsule does not comprise an inert gas. In some instances, the inert gas comprises an elemental gas, a compound gas, a noble gas, helium, neon, argon, krypton, xenon, oganesson, compounds of noble gas, purified argon, purified nitrogen, nitrogen or any combination thereof. In some instances, the inert gas comprises nitrogen. In some cases, the inert gas within a capsule can comprise at least about: 75%, 80%, 85%, 90%, or 95% of the gas on a volume-to-volume basis. In some instances, the capsule further comprises air in the volume not occupied by the powdery pharmaceutical composition.

In some cases, the same active ingredients can be independently separated into the inner capsule, the outer capsule, or both of a capsule-in-capsule composition. In some cases, different active ingredients can be independently separated into the inner capsule, the outer capsule, or both of a capsule-in-capsule composition. For example, an active ingredient comprising a substantially encapsulated cell can be comprised in in an inner capsule and the outer capsule can a different substantially encapsulated cell. In some cases, active compounds in the inner, outer, or both capsules can comprise an excipient such as lactose, microcrystalline cellulose, cellulose, mannitol, sorbitol, starch, starch glycolate, hydroxypropyl methylcellulose, hydroxypropyl methylcellulose acetate succinate, a cyclodextrin, maltodextrin, croscarmellose sodium, honey, corn starch, carrageenan, sorbitol, maltitol, glucose, a pharmaceutically acceptable salt of any of these. In some cases, the active ingredients can be microencapsulated as described herein. After the microencapsulation process, the suspension can be spray dried to create a dry powder finished product. In some cases, a dry powdered finished product can be added to a capsule. In some cases, the inner and the outer capsule can have the same amount of an active drug. In some instances, the inner and the outer capsule may have a different amount of an active drug. In some cases, the ratio of the amount of the compound in the inner capsule and the outer capsule (weight to weight) can be about: 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 55:1, 60:1, 65:1, 70:1, 75:1, 80:1, 85:1, 90:1, 95:1 or 100:1. In some cases, the ratio of the amount of the compound in the inner capsule and the outer capsule (weight to weight) can be about 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:31, 1:32, 1:33, 1:34, 1:35, 1:36, 1:37, 1:38, 1:39, 1:40, 1:41, 1:42, 1:43, 1:44, 1:45, 1:46, 1:47, 1:48, 1:49, 1:50, 1:55, 1:60, 1:65, 1:70, 1:75, 1:80, 1:85, 1:90, 1:95 or 1:100.

In some embodiments, the pharmaceutical composition can be contained within a capsule, wherein the capsule can be at least in part be contained within a device which may be a drug delivery device, an inhalation drug delivery device, a diffuser, an inhaler, a metered dose inhaler, a dry powder inhaler, a soft mist inhaler, or any combination thereof. In some instances, the device may be an inhaler. In some cases, a dry powder inhaler does not comprise a propellent. In some cases, a dry powder inhaler does not comprise a chlorofluorocarbon, a hydrofluorocarbon, a fluorocarbon or any combination thereof as a propellent. In some cases, a dry powder inhaler may not be pressurized. In some instances, a dry powder inhaler comprises breathing or inhaling an active ingredient or composition into the lungs. In some instances, a dry powder inhaler can be breath-activated, wherein when a subject breathes in through an inhaler, the inhaler releases particles (e.g., an active ingredient, excipient or both) which travel throughout the respiratory system. In some cases, a capsule can contain an active ingredient which can be pierced to release the particles prior to inhalation through a dry powder inhaler. In some instances, particle size and aerodynamics can affect travel throughout the respiratory system.

In some embodiments, the pharmaceutical composition can be contained within a capsule, wherein the capsule can be at least in part contained within the device. In some embodiments, the pharmaceutical composition can be contained within a capsule, wherein the capsule can be at least in part contained within the device, and wherein the device contains a sharp surface configured to puncture or slice the capsule. In some embodiments, the pharmaceutical composition can be contained within a capsule, wherein the capsule can be at least in part contained within the device, and wherein the device contains a sharp surface configured to puncture or slice the capsule, and wherein, prior to administrating, the device can be actuated such that the sharp surface punctures or slices the capsule. In some cases, the device can be an inhaler.

In some embodiments, an inhaler unit can be re-used via a process comprising replacing a spent capsule with a new capsule containing a powdery pharmaceutical composition. In some instances, a component of the inhaler unit configured to at least in part hold the capsule can be temporarily at least partially separable from the inhaler unit. In some instances, the capsule can be at least partially visible via an at least partially transparent material present in the inhaler unit.

### Delivery of the Pharmaceutical Composition for Treatment of Diseases

In some embodiments, the administration of the pharmaceutical composition, a supplement, or the second therapeutic can be administered orally, intra nasally, intra ocular, anally, by injection, intra venously, intra muscularly, subcutaneously, intra peritoneally, trans dermally, or any combination thereof.

In some embodiments, the administration of the pharmaceutical composition can be by inhalation. In some instances, inhalation can be oral inhalation, intra nasal administration, or any combination thereof. In some cases, administration can be oral ingestion of a beverage or a food. In some instances, the powdery pharmaceutical composition can be inhaled into human lungs. In some cases, at least a portion of the excipient can deposit on the oropharynx. In some instances, the powdery pharmaceutical composition, when inhaled into the lungs, provides a time to peak plasma concentration (Tmax) of the active ingredient or the salt thereof. The time to peak plasma concentration (Tmax) of the active ingredient or the salt thereof can range from about 1 minute to about one hour. In some instances, the time to peak plasma concentration (Tmax) of the active ingredient or the salt thereof can range from about 1 minute to about ten minutes.

In some embodiments, administering can be by oral ingestion, topical application, or inhalation. In some instances, administering can comprise oral ingestion and the oral ingestion can comprise oral ingestion of a food, a liquid, a gel, a capsule, or any combination thereof. In some instances, administering can comprise topical application and the topical application can comprise topical application of a lotion, a tincture, a balm, a cream, an oil, a gel, a butter, a liquid, a spray, an ointment, a paste, a jelly, or any combination thereof. In some instances, administering can comprise inhalation and the inhalation can comprise inhalation by a diffuser, an inhaler, a nebulizer, or any combination thereof. In some instances, administering can comprise inhalation and the inhalation can comprise inhalation by a diffuser. In some instances, administering can comprise inhalation and the inhalation can comprise inhalation by a nebulizer. In some instances, administering can comprise inhalation and the inhalation can comprise inhalation by an inhaler. In some cases, administration can comprise intra ocular administration, rectal administration, administration by injection, intra venous administration, intramuscular administration, subcutaneous administration, intra peritoneal administration, trans dermal administration, or any combination thereof. In some cases, a cellular material can be injected near joints. In some cases, the at least partially encapsulated cells, at least partially encapsulated one or more cellular components, or both can be administered by an inhaler, a capsule-in-capsule, a capsule, or any combination thereof. In some cases, cells can be contacted with the skin. In some cases, cells can be placed into small indentations in the skin.

In some embodiments, administering can be performed at least about: 1 time per day, 2 times per day, 3 times per day, 4 times per day, 5 times per day, 6 times per day or more than 6 times per day. In some cases, administering can be performed daily, weekly, monthly, or as needed. In some instances, administering can be conducted one, twice, three, or four times per day. In some cases, administration can be provided by a subject (e.g., the patient), a health care provider, or both.

In some embodiments, administering can be performed for about: 1 day to about 8 days, 1 week to about 5 weeks, 1 month to about 12 months, 1 year to about 3 years, 3 years to about 10 years, 10 years to about 50 years, 25 years to about 100 years, or 50 years to about 130 years. In some instances, the composition can be administered as needed, or for about: one day, two days, three days, four days, five days, six days, a week, two weeks, three weeks, four weeks, a month, two months, three months, four months, five months, six months, seven months, eight months, nine months, ten months, eleven months, a year, or chronically.

Also disclosed herein are kits comprising the pharmaceutical composition contained at least in part in packaging. Also disclosed herein are methods of making kits comprising a pharmaceutical composition contained at least in part in packaging. In some cases, a kit can comprise the encapsulated cells, the encapsulated one or more cellular components or both and a container. In some cases, a kit can comprise a supplement disclosed herein.

Also disclosed herein are methods of treating or preventing a disease comprising treating or preventing the disease or condition by administering a therapeutically effective amount of the powdery pharmaceutical composition. In some cases, the powdery pharmaceutical composition can comprise an encapsulated cellular material. Also disclosed herein are methods of treating or preventing a disease comprising treating or preventing the disease or condition by administering a therapeutically effective amount of the powdery pharmaceutical composition. In some instances, the disease can comprise treating or preventing a disease or condition selected from the group consisting of: a cancer, an anxiety, pruritus (itching), cognitive function, Alzheimer's disease, a chronic pain, pain management, multiple sclerosis, side effects of chemotherapy, AIDS, HIV, a neurodegenerative disorder, Tourette syndrome, cervical dystonia, a sleep disorder, an appetite disorder, a nausea associated with chemotherapy, a nausea, anorexia, spinal cord injury, glaucoma, an epilepsy, a seizure, an asthma, a substance dependency disorder (e.g. alcohol, cocaine, amphetamine, opioid), a psychiatric symptom, an autoimmune disease, an inflammation, and any combination thereof. In some cases, a powdery pharmaceutical composition can be administered as a sleep aide, an appetite stimulant, for drug/alcohol dependency withdrawal or a combination thereof. In some instances, a cancer can be a breast cancer, a brain cancer, a tumor, a cervical cancer, a lung cancer, a prostate cancer, a pancreatic cancer, or any combination thereof. In some cases, a cancer can be a sarcoma, a melanoma, a lymphoma, a leukemia, or a combination thereof. In some cases, a disease can comprise neuropathic pain, pain, opioid addiction, opioid overdose, a heart disease, a hypertension, a sleep disorder, Guillain-Barré syndrome, Wilke's syndrome, a brain tumor, a human papillomavirus (HPV) infection, a brain injury (e.g. a traumatic brain injury), a depression, inflammation, Huntington's Disease, emesis, osteoporosis, schizophrenia, a cardiovascular disease, obesity, an infectious disease (bacterial, fungal, or viral), a coronavirus infection, COVID-19, a metabolic syndrome-related disease, an arthritis, fibromyalgia, a dementia, Parkinson's disease or any combination thereof. In some cases, an arthritis can comprise osteoarthritis, fibromyalgia, rheumatoid arthritis, psoriatic arthritis, juvenile arthritis, gout, lupus. In some cases, a disease or condition can comprise pain, such as a chronic pain or an acute pain associated with an arthritis. In some cases, a disease or condition can comprise a pain associated with HIV, such as a chronic pain, an acute pain, or both. In some cases, a disease or condition can comprise inflammation associated with HIV. In some cases, a disease can comprise sickle cell disease. In some instances, sickle cell disease can comprise sickle cell anemia, sickle hemoglobin-C disease, sickle beta-plus thalassemia or sickle beta-zero thalassemia. In some cases, a disease or condition can comprise a pain (e.g., an acute pain or a chronic pain) associated with sickle cell anemia, sickle hemoglobin-C disease, sickle beta-plus thalassemia or sickle beta-zero thalassemia. In some cases, a disease or condition can comprise inflammation associated with sickle cell anemia, sickle hemoglobin-C disease, sickle beta-plus thalassemia or sickle beta-zero thalassemia. In some cases, a composition described herein can alleviate symptoms associated with a disease. For example, a composition described herein can alleviate anemia, fatigue, pain, swelling (e.g., of hands and/or feet), infections, delayed growth, vision problems or any combination thereof.

In some embodiments, an encapsulated cellular material disclosed herein can be used to replace cells damaged by a chemotherapy or a disease. In some cases, a cellular material can be used to treat a cancer such as a leukemia, a lymphoma, a neuroblastoma, a multiple myeloma, a breast cancer, a brain cancer, a tumor, a cervical cancer, a lung cancer, a prostate cancer, or a pancreatic cancer. In some cases, an encapsulated cellular material can be used to treat a blood related disease, a degenerative disease, a heart failure, a spinal cord injury, Type 1 diabetes, Type 2 diabetes, Parkinson's disease, Alzheimer's disease, amyotrophic lateral sclerosis, a stroke, a burn, a skin damage (such as a cut, a scrape, an infection, a wound), an osteoarthritis, an arthritis, a diabetic cognitive dysfunction in type 2 diabetes, astrogliosis associated with diabetes (for example in type 2 diabetes), a depression, a schizophrenia, a genetic disease, a chronic obstructive pulmonary disease (COPD), a chronic bronchitis, an emphysema, a reactive airway disease, an infectious disease (e.g., bacterial parasitic, fungal, or viral), a muscular injury, a skeletal injury, an inflammatory disease, an allergy, a neuronal injury. In some cases, an encapsulated cellular material can be used to treat skin damage and/or tissue damage, for example, a puncture, a laceration, a pressure injury, an incision, an abrasion, a thermal injury (e.g., a burn), a chemical injury, or a combination thereof. In some cases, an encapsulated cellular material can be used to correct organ functionality. In some cases, an encapsulated cellular material can be used to treat ageing. In another example, an encapsulated cellular material can be used to restore damaged tissue, restore damaged nerve cells, restore damaged organs, or any combination thereof. In some cases, an encapsulated cellular material can be used in regenerative medicine. In some cases, regenerative medicine can comprise decreasing the effects of ageing for example by increasing skin elasticity and/or reducing the number, the depth, or the appearance of wrinkles.

In some cases, the cellular material (e.g., least partially encapsulated cells, at least partially encapsulated one or more cellular components, or both) can prevent or slow the development of lung diseases such as chronic bronchitis, emphysema, fibrosis, and chronic obstructive pulmonary disease (COPD). In some cases, the cellular material can further comprise a further active ingredient or salt thereof.

In some embodiments, prior to treating, a subject may have been diagnosed with the disease. In some cases, treatment can comprise diagnosing a subject with a disease. In some instances, the subject may be a human, a man, a woman, an individual over 18 years of age, an individual under 18 years of age, or any combination thereof. In some instances, a subject can be from about 1 day to about 10 months old, from about 9 months to about 24 months old, from about 1 year to about 8 years old, from about 5 years to about 25 years old, from about 20 years to about 50 years old, from about 40 years to about 80 years old, or from about 50 years to about 130 years old. In some instances, a method can further comprise diagnosing a subject as having the disease. In some instances, a diagnosing can comprise employing an in vitro diagnostic. In some instances, the in vitro diagnostic can be a companion diagnostic.

In some embodiments, a diagnosis can comprise a physical examination, a radiological image, a blood test, an antibody test, or any combination thereof. In some instances, a diagnosis can comprise a radiological image and the radiological image can comprise: a computed tomography (CT) image, an X-Ray image, a magnetic resonance image (MRI), an ultrasound image, or any combination thereof.

In some instances, a method can further comprise administering a second therapy to the subject. In some cases, a second therapy can be encapsulated. In some cases, a second therapy may not be be encapsulated. In some cases, a second therapy can be administered concurrently or consecutively. In some instances, a second therapy can comprise acetaminophen, a corticosteroid, an opioid, a nonsteroidal anti-inflammatory drug (NSAID), a COX-2 selective NSAID, a COX-2 inhibitor, methotrexate, hydroxychloroquine, prednisone, cortisone, a biological response modifier, a salt thereof, or any combination thereof. In some instances, a second therapy can comprise a biological response modifier and the biological response modifier can comprise: abatacept, adalimumab, adalimumab-atto, anakinra, certolizumab pegol, etanercept, etanercept-szzs, golimumab, infliximab, infliximab-dyyb, rituximab, sarilumab, tocilizumab, a biologically active fragment of any of these, a salt of any of these, or any combination thereof. In some instances, the second therapy can comprise a nonsteroidal anti-inflammatory drug and the nonsteroidal anti-inflammatory drug can comprise naproxen, ibuprofen, a salt of any of these, or any combination thereof. In some instances, a NSAID can comprise aspirin, diflunisal, dexibuprofen, oxaprozin, fenoprofen, indomethacin, tolmetin, celecoxib, clonixin, ketoprofen, salts thereof, or any combination thereof. In some instances, a COX-2 inhibitor can comprise etoricoxib, celecoxib, rofecoxib, valdecoxib, a salt thereof, or any combination thereof. In some cases, an active ingredient (e.g., cellular material) can be combined with additional cellular material. In some instances, a composition can comprise an excipient, a diluent, a carrier, or any combination thereof.

In some embodiments, a bronchodilator can be administered before, concurrently or after administration of the powdery pharmaceutical composition described herein (e.g., an encapsulated cellular material). In some cases, a bronchodilator can comprise a long acting or a short acting bronchodilator. In some instances, a bronchodilator can comprise a beta-2 antagonist, an anticholinergic, a xanthine derivative or a combination thereof. In some cases, a short acting bronchodilator can comprise albuterol, levalbuterol, pirbuterol, or a combination thereof. In some cases, a long-acting bronchodilator can comprise salmeterol, formoterol, aclidinium, tiotropium, umeclidinium, or a combination thereof.

In some instances, before the administration of a composition described herein a patient can be given a test to evaluate lung function. In some cases, a lung function test (e.g. pulmonary function test) can comprise spirometry, body plethysmography, a lung volume test, a lung diffusion capacity assay, a pulse oximetry test, a forced expiatory volume test, an arterial blood gas test, a fractional exhaled nitric oxide test, or any combination thereof.

In some embodiments, about: 1×10³, 1×10⁴, 1×10⁵, 1×10⁶, 1×10⁷, 1×10⁸, 1×10⁹, 1×10¹⁰ or about 1×10¹¹ cells or cellular components can be administered to a subject. In some instances, more than about: 1×10³, 1×10⁴, 1×10⁵, 1×10⁶, 1×10⁷, 1×10⁸, 1×10⁹, 1×10¹⁰ or about 1×10¹¹ cells or cellular components can be administered to a subject. In some instances, less than about: 1×10³, 1×10⁴, 1×10⁵, 1×10⁶, 1×10⁷, 1×10⁸, 1×10⁹, 1×10¹⁰ or about 1×10¹¹ cells or cellular components can be administered to a subject. In some instances, about: 1×10³ to about 1×10⁷, 1×10³ to about 1×10⁵, 1×10⁴ to about 1×10⁸, 1×10⁶ to about 1×10⁹, 1×10⁴ to about 1×10⁶, 1×10⁵ to about 1×10⁸, 1×10⁷ to about 1×10¹⁰, or about 1×10⁸ to about 1×10¹² cells or cellular components can be administered to a subject.

In some embodiments, the composition can be administered so that the active ingredient or the pharmaceutically acceptable salt thereof in the unit dose ranges from about: 500 μg (micrograms) to about 1000 mg, 10 μg to about 50 μg, 40 μg to about 90 μg, 80 μg to about 120 μg, 100 μg to about 150 μg, 140 μg to about 190 μg, 150 μg to about 220 μg, 200 μg to about 250 μg, 240 μg to about 300 μg, 290 μg to about 350 μg, 340 μg to about 410 μg, 400 μg to about 450 μg, 440 μg to about 500 μg, 500 μg to about 700 μg, 600 μg to about 900 μg, 800 μg to about 1 mg (milligram), 1 mg to about 5 mg, 1 mg to about 10 mg, 5 mg to about 15 mg, 12 mg to about 25 mg, 20 mg to about 50 mg, 40 mg to about 80 mg, 70 mg to about 100 mg, 90 mg to about 150 mg, 125 mg to about 250 mg, 200 mg to about 500 mg, 400 mg to about 750 mg, 700 mg to about 900 mg, or from about 850 mg to about 1000 mg. In some cases, the unit dose range can be more than about or equal to: 10 μg, 25 μg, 50 μg, 75 μg, 100 μg, 150 μg, 200 μg, 250 μg, 300 μg, 350 μg, 400 μg, 450 μg, 500 μg, 550 μg, 600 μg, 650 μg, 700 μg, 750 μg, 800 μg, 850 μg, 900 μg, 950 μg, 1000 μg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, or 25 mg. In some cases, the unit dose range can be less than about: 10 μg, 25 μg, 50 μg, 75 μg, 100 μg, 150 μg, 200 μg, 250 μg, 300 μg, 350 μg, 400 μg, 450 μg, 500 μg, 550 μg, 600 μg, 650 μg, 700 μg, 750 μg, 800 μg, 850 μg, 900 μg, 950 μg, 1000 μg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, or 25 mg. Methods of administration and details thereof are shown in Table 1.

TABLE 1
Routes of Administration

| Route of | Particle | | |
|---|---|---|---|
| Administration | Size | Notes | Delivery |
| Intranasal | About 10 | Reconstituted dry powder | The dry powder can be stored |
| | μm to | with Water for Injection | in sterile clear glass vial with |
| | about 20 | (“WFI”) and saline and | Nitrogen and hermetically |
| | μm | delivered in a nasal spray | sealed. The hermetically |
| | | | sealed container can have a |
| | | | pull pop top. There can be a |
| | | | silicone stopper to allow the |
| | | | introduction of Water for |
| | | | Injection (“WFI”) via a |
| | | | syringe. |
| Oral Inhalation | About 1.0 | Delivered via a dry | The dry powder can be |
| | μm to | powder inhaler. | placed inside a capsule with |
| | about 5.0 | | appropriate excipient and |
| | μm | | sealed in a hermetically seal |
| | | | container. The powder can |
| | | | be delivered using a Dry |
| | | | Powder inhaler. |
| Oral | About 70 | Delivered in a capsule-in- | The dry powder can be |
| | μm to | capsule to the intestines. | placed inside a capsule with |
| | about 100 | (Enteric coated to bypass | appropriate excipient and the |
| | μm | the stomach) | capsules can be placed in a |
| | | | hermetically seal container. |
| | | | The capsule can be taken |
| | | | orally with water. |
| Intravenous/ | Less than | Reconstituted dry powder | The dry powder can be stored |
| Injection | about 40 | with WFI and injected to | in sterile clear glass vial with |
| Reconstituted | microns | target area. | Nitrogen and hermetically |
| Dry Powder | | | sealed. The hermetically |
| | | | sealed container can have a |
| | | | pull pop top. There can be a |
| | | | silicone stopper to allow the |
| | | | introduction of Water for |
| | | | Injection (“WFI”) via a |
| | | | syringe. |
| Eye Drop | Less than | Reconstituted dry powder | The dry powder can be stored |
| Reconstituted | about 40 | with WFI (and saline) and | in sterile clear glass vial with |
| Dry Powder | microns | administered with eye | Nitrogen and hermetically |
| | | dropper. | sealed. There can be WFI |
| | | | (and saline) to be mixed to |
| | | | enable delivery of the eye |
| | | | drops. |
| Topical | About 10 | Topical administration by | The dry powder can be stored |
| administration | μm to | a cream, a balm, a lotion, | in a sterile tube or dispenser |
| | about 100 | a tincture, a dry powder, a | and sealed. The dry powder |
| | microns | liquid, a gel, a paste, an | may be mixed with an |
| | | ointment, a patch, a foam, | excipient and/or carrier. The |
| | | a shake lotion, a solid, a | dry powder can be mixed into |
| | | spray, or a combination | a solution such as a cream, a |
| | | thereof | balm, a lotion and placed into |
| | | | a sterile tube. |

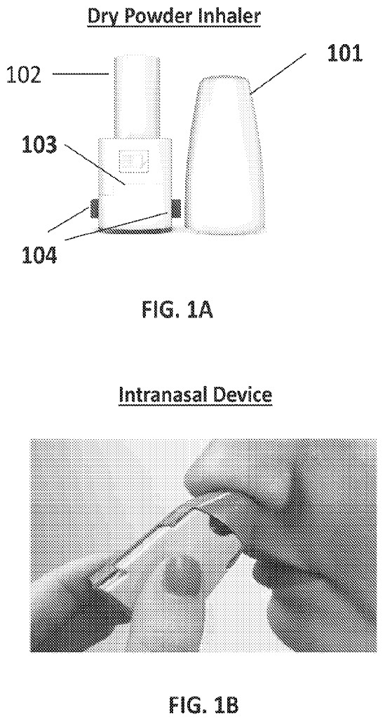

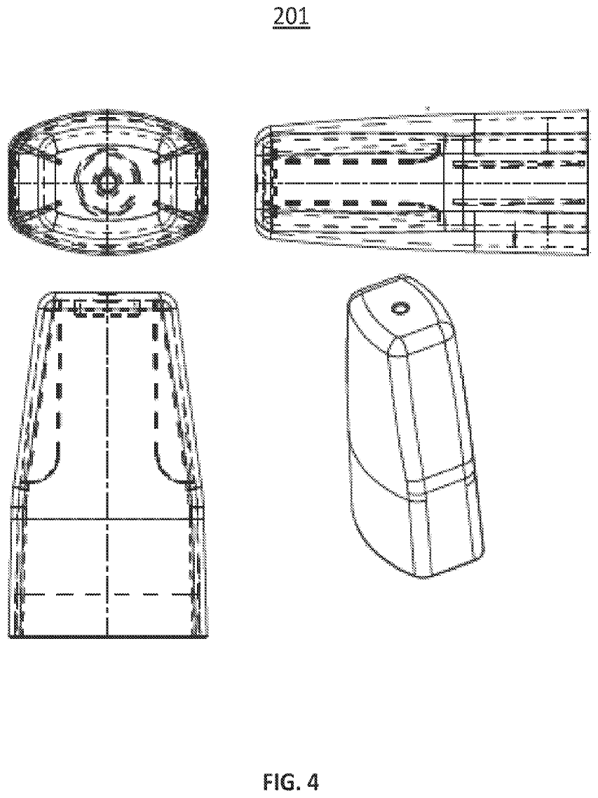

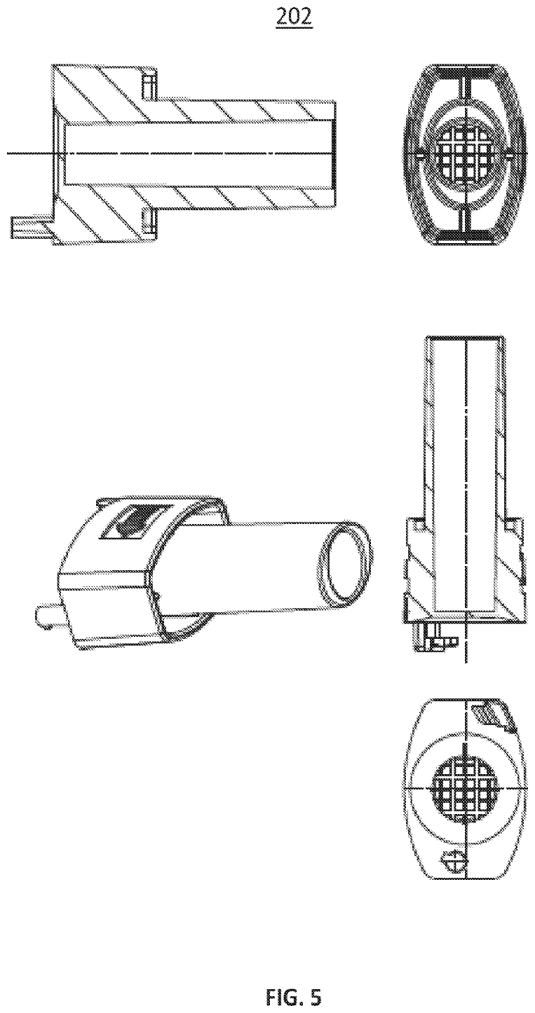

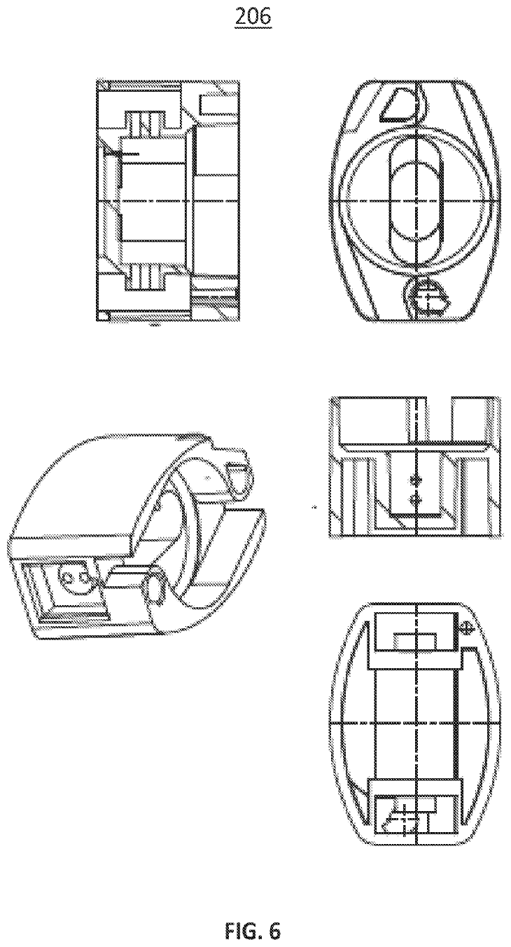

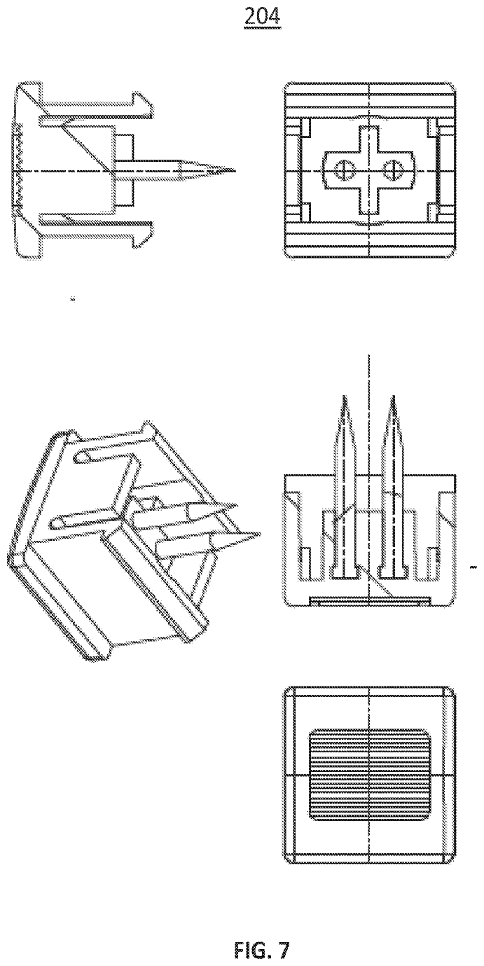

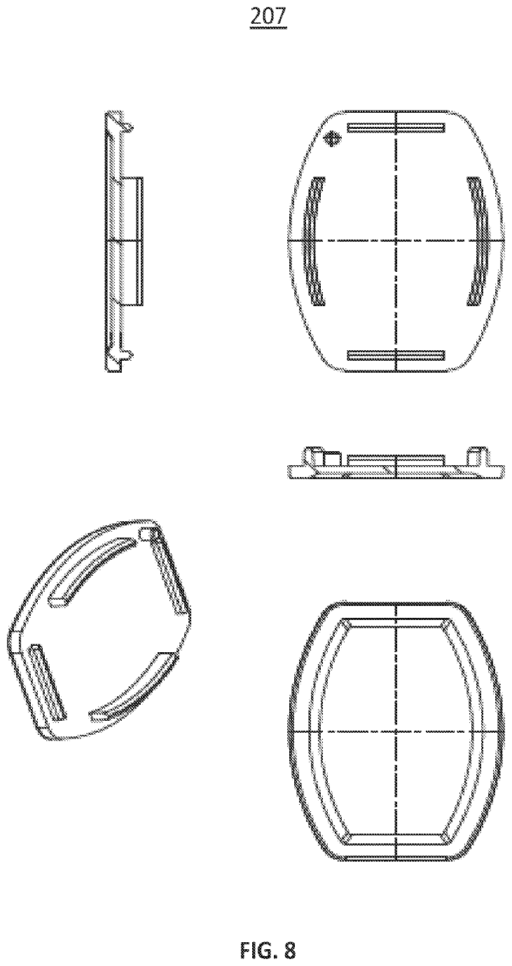

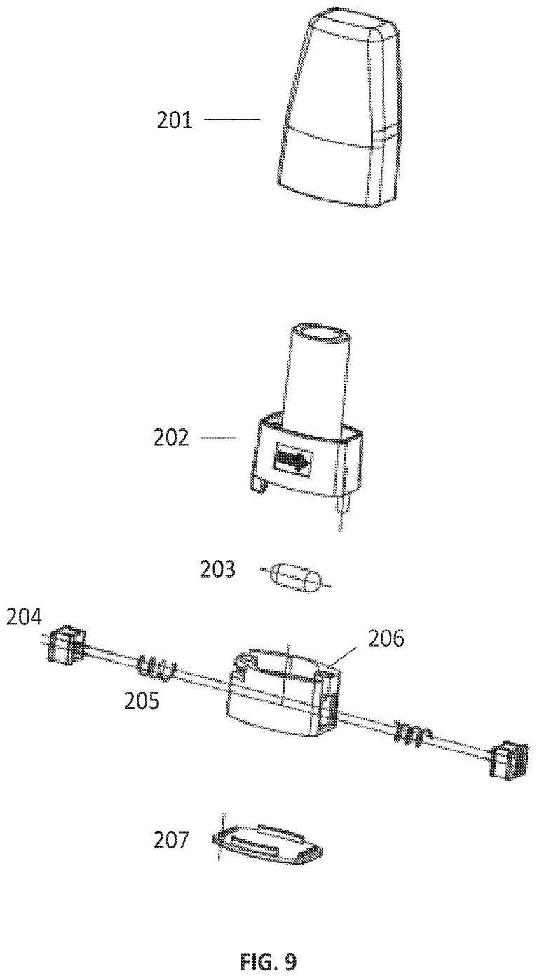

Referring to FIG. 1, FIG. 1A shows a dry powder inhaler device for delivery of powdery pharmaceutical compositions to the lung alveolar. The inhaler device can comprise a protective cap shown in FIG. 4, a rotatable top comprising a mouthpiece shown in FIG. 5, a lower base chamber receptacle for placing a pharmaceutical capsule shown in FIG. 6, a lateral button for mechanically piercing a capsule with a sharp surface while inside the chamber show in FIG. 7, and a chamber aerially connected to the mouthpiece permitting inhalation of capsule contents. The dry powder inhaler device can comprise a base plate as shown in FIG. 8. FIG. 9 shows a dry powder inhaler device with a protective cap, a rotatable comprising a mouthpiece, a lower base chamber for piercing a pill and a base plate.

### EXAMPLES

The following examples are included for illustrative purposes only and are not intended to limit the scope of the disclosure.

### Example 1

The powdery pharmaceutical composition described herein is administered by a dry powder inhaler or by a nasal inhaled device. Referring to FIG. 1A, FIG. 1A shows a dry powder inhaler device for delivery of a powdery pharmaceutical composition described herein to the lung alveolar. The inhaler device comprises: a protective cap 101, a rotatable top comprising a mouthpiece 102, a lower base chamber receptacle for placing the pharmaceutical capsule 103, lateral buttons for mechanically piercing the capsule with a sharp surface while inside the chamber 104, wherein the chamber is aerially connected to the mouthpiece permitting inhalation of capsule contents. FIG. 1B shows the nasal administration by a nasal inhaled device of a powdery pharmaceutical composition in a human subject. The composition is inhaled via the nares after the capsule containing the composition is pierced within the nasal inhaled device.

### Example 2

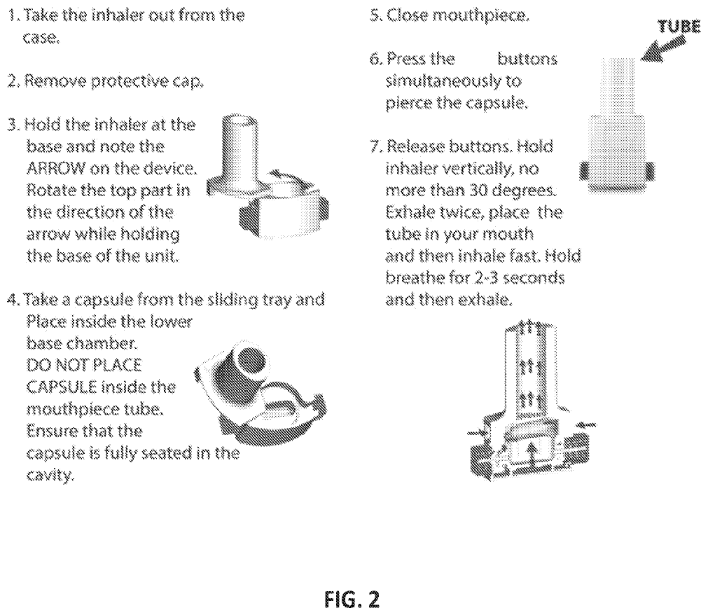

The method of using an inhaler device for the administration of a dry powdery pharmaceutical composition is shown in FIG. 2. The process for administration of the dry powdery pharmaceutical composition comprises 7 steps. Step 1: The inhaler is removed from the case. Step 2: The protective cap is removed. Step 3: The inhaler is held at the base and the top part is rotated in the direction of the arrow while the base of the unit is held. Step 4: A capsule is placed inside the lower base chamber cavity. Step 5: The mouthpiece is closed. Step 6: The buttons are pressed simultaneously to piece the capsule. Step 7: The buttons are released. The inhaler is held vertically, e.g., no more that about 30 degrees. The subject exhales twice before placing the tube in their mouth. The subject inhales quickly and holds their breath for about 2-3 seconds before exhaling.

### Example 3

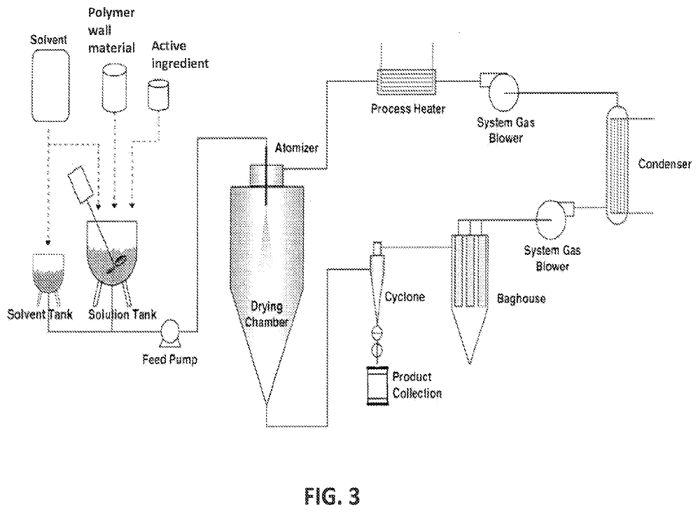

The active encapsulated ingredient (e.g., an exosome) in a dry powdery pharmaceutical composition described herein was manufactured by a spray drying system. FIG. 3 shows a spray drying manufacturing system for microencapsulated cellular material comprising a closed spray drying chamber which receives a solution comprising a polymer wall material in a suitable solvent mixed with droplets comprising the active ingredient (e.g., an exosome). Prior to spray drying, a polymer wall material (trehalose) was dissolved in a solvent (water). The active ingredient (an exosome) was mixed thoroughly with the solvent and wall material by low frequency mixing. The microencapsulated liquid suspension was fed into the atomizer. There is a spray nozzle at the top of the chamber, where the suspension was atomized with an inert gas. The atomizer can be a two component (air/nitrogen and liquid), rotary, hydraulic (pressure-type), or ultrasonic nozzle types that distributes the suspension into fine droplets controlled by the atomizer pressure to achieve proper particle size for optimum absorption for the route of administration. The liquid feed was converted into small droplets by the atomizer and sprayed into a hot gas path (less than 35° C. to maintain stability of the exosome) that flash dried the droplets into solid particles. The aqueous solvent was evaporated and the particles were collected at the exit chamber. The drying chamber produced uniform fine particles that maintain tight particle size distribution. The particles were separated from the drying gas using a cyclone separator or filter bag. The spray drying technology controlled the particle size and particle size distribution.

### Example 4

Pig umbilical cord stem cells were formulated in a dry powdery composition and manufactured by a spray drying system described above. Briefly, the closed spray drying chamber received a solution comprising methyl cellulose, ethyl cellulose, poly vinyl alcohol (PVA), or povidone (poly vinyl pyrrolidone) dissolved in a solvent (e.g., a water and ethanol mix). The solution was placed in a beaker where the solvent and encapsulation material were mixed. The pig umbilical cord stem cells were slowly added to the solution. The microencapsulated liquid suspension was fed into the atomizer and spray dried as described above.

### Example 5

A male subject is diagnosed with chronic obstructive pulmonary disease (COPD). The subject is prescribed a dosing regimen of a pharmaceutical composition. The pharmaceutical composition comprises encapsulated mesenchymal stem cells which are processed to a dry powder using the methods described herein (e.g., spray drying). The cellular dry powder is mixed with a lactose powder and encapsulated. The encapsulated cells are packaged in a capsule and are administered intranasally with an inhaler. The dosing regimen comprises an effective amount (e.g., 0.5 mg, 1.0 mg, 2.5 mg or 5.0 mg) of encapsulated cells to treat the disease.

### Example 6

A subject is diagnosed with amyotrophic lateral sclerosis (ALS). The subject is prescribed a dosing regimen of a pharmaceutical composition. The pharmaceutical composition comprises encapsulated very small embryonic stem cells (VSELS) and induced pluripotent stem cells, which are processed separately to a dry powder using the methods described herein (e.g., spray drying). The VSELS dry powder is mixed with an excipient and encapsulated into a first capsule. The first capsule is sealed and coated with a time-release coating. The pluripotent stem cell dry powder is mixed with an excipient and placed into the second capsule. Additionally, the first capsule is placed into the second capsule. The second capsule is sealed and coated with a pH dependent coating. The capsule-in-capsule is administered orally.

### Example 7

The powdery pharmaceutical composition described herein is administered by a dry powder inhaler. Referring to FIG. 9, FIG. 9 shows a dry powder inhaler device for delivery of a powdery pharmaceutical composition described herein to the lung alveolar. The inhaler device comprises: a protective cap 201, a rotatable top comprising a mouthpiece 202, a lower base chamber receptacle 206 for placing the pharmaceutical capsule 203, lateral buttons for mechanically piercing the capsule with a sharp surface 204 while inside the chamber with the use of a spring 205, wherein the chamber is aerially connected to the mouthpiece permitting inhalation of capsule contents. The baseplate 207 is fitted to the lower base chamber receptacle.

While certain aspects of the present disclosure have been shown and described herein, it will be obvious to those skilled in the art that such aspects are provided by way of example only. Numerous variations, changes, and substitutions will now occur to those skilled in the art without departing from the disclosure. It should be understood that various alternatives to certain aspects within the disclosure described herein may be employed in practicing the methods and compositions, and systems described herein. It is intended that the following claims define the scope of the disclosure and that methods and structures within the scope of these claims and their equivalents be covered thereby.

What is claimed is:

1. A powdery composition, comprising:
a plurality of spray dried particles, each particle of the plurality of spray dried particles comprising a eukaryotic cell, substantially encapsulated in a coating material,
wherein:
(i) each particle of the plurality of spray dried particles individually has a particle diameter ranging from about 5 micrometers to about 100 micrometers, as measured by a particle analyzer using laser diffraction,
(ii) the eukaryotic cell is a human cell,
(iii) about 1% to 100% of the eukaryotic cells in the plurality of particles are alive, and
(iv) the coating material comprises a hydroxypropyl methylcellulose (HPMC), a hydroxypropyl methylcellulose acetate succinate (HPMCAS), a cyclodextrin, a maltodextrin, a povidone, a copovidone, a trehalose, or any combination thereof.

2. The powdery composition of claim 1, further comprising a pharmaceutically acceptable: excipient, diluent, or carrier.

3. The powdery composition of claim 2, comprising the pharmaceutically acceptable excipient, wherein the pharmaceutically acceptable excipient is in particle form, and wherein at least a portion of the particles of the pharmaceutically acceptable excipient individually have a particle diameter ranging from about 50 micrometers to about 200 micrometers, as measured by a particle analyzer using laser diffraction.

4. The powdery composition of claim 3, wherein the plurality of spray dried particles and the pharmaceutically acceptable excipient are admixed into a substantially homologous mixture.

5. The powdery composition of any one of claims 2-4, comprising the pharmaceutically acceptable excipient, wherein the pharmaceutically acceptable excipient comprises a carbohydrate, an alginate, povidone, a carbomer, a flavor, a natural gum, a silicone, an alcohol, a butter, a wax, a fatty acid, a preservative, a pharmaceutically acceptable salt of any of these, or any combination thereof.

6. The powdery composition of any one of claims 1-5, comprising from about 1 mg to about 200 mg of eukaryotic cells.

7. The powdery composition of any one of claims 1-5, comprising from about 10³ eukaryotic cells to about 10¹² eukaryotic cells.

8. The powdery composition of any one of claims 1-7, wherein the eukaryotic cell comprises an adult stem cell, an embryonic stem cell, a blood stem cell, a neural stem cell, an epithelial stem cell, a skin stim cell, mesenchymal stem cell, a very small embryonic-like stem cell (VSELs), a peripheral blood stem cell, (blastomeres), an induced pluripotent stem cell, an epithelial cell, an endothelial cells a fat cell, a white blood cell, a nerve cell, a platelet, a skeletal cell, a cartilage cell or a red blood cell.

9. The powdery composition of any one of claims 1-8, further comprising a substantially encapsulated different eukaryotic cell.

10. The powdery composition of any one of claims 1-9, wherein the eukaryotic cell comprises at least about 1% by weight to about 99% of the overall powdery pharmaceutical composition.

11. A powdery composition, comprising:
a plurality of spray dried particles, each particle of the plurality of spray dried particles comprising a cellular component or derivative thereof, substantially encapsulated in a coating material,
wherein
(i) each particle of the plurality of spray dried particles individually has a particle diameter ranging from about 1 micrometer to about 100 micrometers or about 1 micrometer to about 10 micrometers, as measured by a particle analyzer using laser diffraction, and
(ii) the coating material comprises a hydroxypropyl methylcellulose (HPMC), a hydroxypropyl methylcellulose acetate succinate (HPMCAS), a cyclodextrin, a maltodextrin, a povidone, a copovidone, a trehalose, or any combination thereof.

12. The powdery composition of claim 11, wherein the cellular component or derivative thereof comprises a protein having at least 75% homology and at least 75% length to SEQ ID NO: 1.

13. The powdery composition of claim 12, comprising the cellular component or derivative thereof wherein the cellular component comprises at least 90% sequence homology to SEQ ID NO: 1.

14. The powdery composition of any one of claims 1-13, which is contained within a capsule or a container.

15. The powdery composition of any one of claims 1-14, wherein the plurality of spray dried particles is encapsulated by one or more additional coating materials.

16. The powdery composition of claim 15, wherein the coating materials are the same coating material.

17. The composition of claim 15, wherein the coating materials are different coating materials.

18. The powdery composition of any one of claims 1-17, wherein the powdery pharmaceutical composition is a powdery pharmaceutical composition optionally in unit dose form.

19. The powdery composition of any one of claims 1-18, wherein the powdery formulation is for ocular use.

20. The powdery composition of any one of claims 1-18, wherein the powdery formulation is for intravenous, or subcutaneous use.

21. The powdery composition of any one of claims 1-18, wherein the powdery formulation is for topical use.

22. The powdery composition of any one of claims 1-18, wherein the powdery formulation is for oral use.

23. The powdery composition of claim 22, wherein the plurality of spray dried particles are at least partially surrounded by a first capsule, a second capsule, or both; and wherein the first capsule is surrounded by the second capsule.

24. The powdery composition of claim 23, wherein the powdery composition further comprises a second active ingredient, or a pharmaceutically acceptable salt thereof in unit dose form.

25. The powdery composition of claim 24, wherein the second active ingredient is at least partially surrounded by the first capsule, the second capsule or both.

26. The powdery composition of claim 24, wherein the second active ingredient comprises particles, and wherein the particles are at least partially encapsulated by a coating material and wherein the particles at least partially encapsulated by the coating material are spray dried.

27. The powdery composition of any one of claims 23-26, wherein the first capsule, the second capsule, or both comprise a capsule coating.

28. The powdery composition of claim 27, wherein the capsule coating at least partially controls active ingredient release.

29. The powdery composition of claim 27, wherein the capsule coating comprises an enteric coating, a time release coating, a pH dependent coating, a delayed release coating, an extended-release coating, or a combination thereof.

30. The powdery composition of any one of claims 23-29, wherein the first capsule and the second capsule are formulated to deliver their contents at different locations in the gastrointestinal system.

31. The powdery composition of any one of claims 23-29, wherein the first capsule and the second capsule are formulated to deliver their contents at about the same location in the gastrointestinal system.

32. The powdery composition of any one of claims 23-31, further comprising adding a capsule band to the first capsule, the second capsule, or both and wherein the capsule band at least partially seals the capsule.

33. The powdery composition of any one of claims 23-32, wherein the first capsule, the second capsule, or both comprise a hydroxypropyl methylcellulose (HPMC).

34. The powdery composition of any one of claims 23-33, wherein the first capsule is size: 000, 00, 0, 1, 2, 3, 4, or 5.

35. The powdery composition of claim 34, wherein the first capsule is size: 1, 2, 3, 4, or 5.

36. The powdery composition of any one of claims 23-33, wherein the second capsule is size: 000, 00, 0, 1, 2, 3, or 4.

37. The powdery composition of claim 36, wherein the second capsule is size: 000, 00, 0, or 1.

38. The powdery composition of any one of claims 23-37, wherein the eukaryotic cell or the cellular component substantially encapsulated in the coating material have a particle diameter ranging from about 10 micrometers to about 100 micrometers or about 20 micrometers to about 80 micrometers.

39. The powdery composition of any one of claims 1-18, wherein the powdery formulation is for inhaled or intranasal use.

40. The powdery composition of claim 39, wherein the powdery formulation is contained within an inhaler unit.

41. The powdery composition of claim 40, wherein the inhaler unit comprises the powdery composition in the capsule.

42. The powdery composition of claim 41, wherein the capsule is about one quarter to about one half, by volume, filled with the powdery composition.

43. The powdery composition of claim 42, wherein a portion of the capsule not containing the powdery composition comprises a gas that at least partially comprises an inert gas.

44. The powdery composition of any one of claims 41-43, wherein the capsule is size: 000, 00, 0, 1, 2, 3, or 4.

45. The powdery composition of claim 44, comprising the capsule, wherein the capsule is size 3.

46. The powdery composition of any one of claims 39-45, wherein the plurality of spray dried particles comprising the eukaryotic cell substantially encapsulated in the coating material individually have a particle diameter ranging from about 5 micrometers to about 30 micrometers, 10 micrometers to about 20 micrometers, or about 5 micrometer to about 15 micrometers.

47. The powdery composition of any one of claims 39-45, wherein the plurality of spray dried particles comprising the cellular component substantially encapsulated in the coating material individually have a particle diameter ranging from about 1 micrometers to about 30 micrometers, 10 micrometers to about 20 micrometers, or about 1 micrometer to about 10 micrometers.

48. A kit comprising the powdery composition of any one of claims 1-47 contained at least in part in a packaging.

49. A method of treating or preventing a disease or condition in a subject in need thereof, comprising treating or preventing the disease or condition by administering a therapeutically effective amount of the powdery composition of any one of claims 1-47 to the subject in need thereof.

50. The method of claim 49, wherein the administering is conducted one, twice, three, or four times per day.

51. The method of claim 49 or 50, wherein the disease or condition is selected from the group consisting of: a blood related disease, a degenerative disease, a heart failure, a spinal cord injury, Type 1 diabetes, Type 2 diabetes, Parkinson's disease, Alzheimer's disease, amyotrophic lateral sclerosis, a stroke, a burn, a skin damage, an osteoarthritis, an arthritis, a diabetic cognitive dysfunction in type 2 diabetes, astrogliosis associated with diabetes, a depression, a schizophrenia, a genetic disease, a chronic obstructive pulmonary disease (COPD), a chronic bronchitis, an emphysema, a reactive airway disease, an infectious disease, a muscular injury, a skeletal injury, an inflammatory disease, an allergy, and a neuronal injury.

52. The method of any one of claims 49-51, wherein the powdery composition is administered as needed, or for about: one day, two days, three days, four days, five days, six days, a week, two weeks, three weeks, a month, two months, three months, four months, five months, six months, seven months, eight months, nine months, ten months, eleven months, a year, or chronically.

53. The method of any one of claims 49-52, wherein an amount of the eukaryotic cell or the cellular material ranges from about 1 mg to about 200 mg.

54. The method of any one of claims 49-52, wherein an amount of the eukaryotic cell or the cellular material ranges from about 1 mg to about 10 mg.

55. The method of any one of claims 49-54, wherein a second therapeutic or pharmaceutically acceptable salt thereof is administered.

56. The method of claim 55, wherein the second therapeutic or a pharmaceutically acceptable salt thereof is administered concurrently or consecutively.

57. The method of claim 55, wherein the second therapeutic or the pharmaceutically acceptable salt thereof is comprised in the powdery formulation.

58. The method of claim 55, wherein the second therapeutic or the pharmaceutically acceptable salt thereof is not comprised in the powdery formulation.

59. The method of any one of claims 49-58, wherein the subject is diagnosed with the disease or condition.

60. The method of claim 59, wherein the diagnosing comprises employing an in vitro diagnostic.

61. The method of claim 60, wherein the in vitro diagnostic is a companion diagnostic.

62. A method of making the powdery composition of any one of claims 1-10, comprising contacting:
the eukaryotic cell and the coating material in a solution, wherein the coating material comprises the hydroxypropyl methylcellulose (HPMC), the hydroxypropyl methylcellulose acetate succinate (HPMCAS), the cyclodextrin, the maltodextrin, the povidone, the copovidone, a trehalose, or any combination thereof and spray drying the eukaryotic cell, the coating material and the solvent to form a substantially encapsulated eukaryotic cell.

63. A method of making the powdery composition of any one of claims 11-12, comprising contacting:
the cellular component and the coating material in a solution, wherein the coating material comprises the hydroxypropyl methylcellulose (HPMC), the hydroxypropyl methylcellulose acetate succinate (HPMCAS), the cyclodextrin, the maltodextrin, the povidone, the copovidone, a trehalose, or any combination thereof and spray drying the cellular component, the coating material and the solvent to form a substantially encapsulated cellular component.

64. A powdery composition, comprising:
a plurality of spray dried particles, each particle of the plurality of spray dried particles comprising a cellular component or derivative thereof, substantially encapsulated in a coating material,
wherein
(i) each particle of the plurality of spray dried particles individually has a particle diameter ranging from about 1 micrometer to about 100 micrometers or about 1 micrometer to about 10 micrometers, as measured by a particle analyzer using laser diffraction,
(ii) the cellular component or derivative thereof comprises a protein having at least 75% homology and at least 75% length to SEQ ID NO: 1,
(iii) the coating material comprises a hydroxypropyl methylcellulose (HPMC), a hydroxypropyl methylcellulose acetate succinate (HPMCAS), a cyclodextrin, a maltodextrin, a povidone, a copovidone, a trehalose, or any combination thereof.
